(19) United States
(12) Patent Application Publication
(10) Pub. No.: US 2016/0115091 A1
(43) Pub. Date: Apr. 28, 2016

(54) Treatment of Carbonaceous Feedstocks

(71) Applicants: Rajai BAHMAN, Denver, CO (US); CIRIS ENERGY, INC., Centennial,, CO (US)

(72) Inventors: Robert Bartek, Centennial, CO (US); Bahman Rejai, Denver, CO (US)

(73) Assignee: CIRIS ENERGY, INC., Centennial, CO (US)

(21) Appl. No.: 14/890,786

(22) PCT Filed: May 14, 2014
(86) PCT No.: PCT/US14/00089

§ 371 Date: Nov. 12, 2015

Related U.S. Application Data
(60) Provisional application No. 61/823,023, filed on May 14, 2013.
(60) Provisional application No. 61/862,717, filed on Aug. 6, 2013.
(60) Provisional application No. 61/929,401, filed on Jan. 20, 2014.

(51) Int. Cl. C07B 33/00; C07D 307/68; C07B 41/08; C07C 51/16

(52) U.S. Cl. CPC C07B 33/00; C07C 51/16; C07D 307/68; C07B 41/08

(57) ABSTRACT

A method for treatment of a carbonaceous feedstock such as coal or black liquor is disclosed. The method comprises heating a mixture of the carbonaceous feedstock, with or without a solubilizing agent, water, and an oxidizing agent to solubilize and oxidize carbonaceous materials. In case of oxidation of black liquor, at least one organic compound comprising from about 2 to about 20 carbon atoms may be obtained. The reaction products may be chemically or physically separated, recycled to the heating step and/or subjected to microbial digestion in order to generate one or more desirable products from the carbonaceous feedstock.

### FIELD OF THE INVENTION

The present invention relates to conversion of insoluble carbonaceous feedstocks to water soluble products. In particular, the present invention is directed to oxidation of the carbonaceous feedstocks to produce valuable chemical products and/or biodegradable substrates, and oxidative steam-stripping of carbonaceous feedstocks, including coal.

Further, the present invention relates to a conversion of organic compounds in pulp mill black liquor. In particular, the present invention is also directed to a method for treating black liquor, comprising treating the black liquor with an oxidizing agent to generate an organic compound comprising from about 2 to about 20 carbon atoms.

### DESCRIPTION OF THE RELATED TECHNOLOGY

Due to energy prices and environmental concerns, various carbonaceous materials, especially those that have previously been considered less suitable for use as fuel, have received renewed attention. These materials may be processed to generate products ranging from usable fuel to raw materials for various industries, such as natural gas, hydrogen, methanol, organic acids, and longer hydrocarbons. For example, carbonaceous materials can be gasified at elevated temperature and pressure to produce a synthesis gas stream that can subsequently be converted to gaseous fuel.

Conversion coal as a carbonaceous material feedstock to valuable liquid fuels and chemicals has been studied and described extensively in prior art. These conversion technologies fall under main categories of hydroliquefaction or direct liquefaction, pyrolysis and gasification. In these processes, coal is depolymerized to varying degrees to its organic constituents with or without oxygen, with or without water. The goal in all these technologies is coal beneficiation by making a mixture of higher value fuels or chemicals or a precursor to desirable fuels or chemicals. However, these processes typically take place either at high temperatures, pressures and/or they use expensive hydrogen and organic solvents.

For example the indirect coal liquefaction (ICL) process consists of a gasification step, at temperatures greater than about 700 degrees Celsius) in the presence of oxygen or air to make syngas (a mix of CO & H₂) followed by at least one catalytic step which converts syngas to liquid hydrocarbons. This is a very capital intensive process.

Direct coal liquefaction process (DCL) on the other hand converts coal into liquids directly, without the intermediate step of gasification, by breaking down its organic structure with application of solvents and catalysts in a high pressure and temperature environment using hydrogen. Since liquid hydrocarbons generally have a higher hydrogen-carbon molar ratio than coals, either hydrogenation or carbon-rejection processes are employed in both ICL and DCL technologies. Both processes require a significant energy consumption and, at industrial scales (thousands of barrels/day), large capital investments.

Generally, the gasification process consists of feeding carbonaceous materials into a heated chamber (the “gasifier”) along with a controlled and/or limited amount of oxygen and optionally steam. In contrast to incineration or combustion, which operates with excess oxygen to produce CO₂, H₂O, SOₓ (including products such as SO, SO₂, SO₃, S₇O₂, S₆O₂, S₂O₂, etc), and NOₓ (including such products as NO, NO₂, N₂O), gasification processes produce a raw gas composition comprising CO, H₂, H₂S, and NH₃. After clean-up, the primary gasification products of interest are H₂ and CO. See Demirbas, “Recovery of Energy and Chemicals from Carbonaceous Materials,” Energy Sources, Part A, vol. 28, pages 1473-1482, 2006.

The carbonaceous materials may also be solubilized to produce valuable starting materials for various industries. U.S. Pat. No. 4,345,098 discloses a process for producing an isomerized benzene carboxylic acid salt by treating a mixture of a carbonaceous material, water, and a water soluble reagent comprising a Group Ia or IIa metal with oxygen under conditions sufficient to convert at least a portion of the aromatic compounds in the carbonaceous material to a benzene carboxylic acid salt of the metal; and isomerizing the benzene carboxylic acid salt by heating without converting the benzene carboxylic acid salt to a benzene carboxylic acid salt of a different Group Ia or IIa metal prior to isomerizing. The benzene carboxylic acid salt is then recovered from the reaction mixture. Their preferred temperature for this process ranges from 200° C. to 350° C. and a pressure of 1700 psig.

U.S. Patent Application Publication No. 2012/0064609 discloses a method for contacting coal or lignocellulosic materials with a composition comprising a pyrophosphate or a derivative thereof. Solubilization of coal or lignocellulosic materials can be carried out in a subterranean formation, in a terrestrial formation or in an ex situ reactor. The method comprises the step of introducing a composition with a pyrophosphate or a derivative thereof into the coal or lignocellulosic materials so as to cause solubilization of the coal or lignocellulosic materials.

U.S. Pat. No. 2,193,337 discloses a process for producing oxalic acid salts by heating carbonaceous materials such as sawdust, woodchips, peat or coal, with oxygen-containing gases at elevated pressures and temperatures in the presence of at least 10 times the weight of carbonaceous material of water and preferably an oxide or hydroxide of an alkali or alkaline earth metal, in an amount of 1.5 to 4 times the weight of feedstock. The oxalic acid, as well as possibly other organic acids such as mellitic acid, benzoic acid, or acetic acid, may then be isolated from the resulting products. The examples in the patent show that a preferred temperature is 180° C., that the pressure should be maintained at 20 atmospheres and that a reaction time of 2 hours can be used.

U.S. Pat. No. 2,786,074 discloses a process for making organic acids from carbonaceous materials. The process oxidizes a carbonaceous material with gaseous oxygen in the presence of an aqueous alkaline solution at elevated temperature (200-270° C.) and pressure (750-1000 psi gauge). The yield of the process may be improved by continuously monitoring the concentration of carbon dioxide and removing excess carbon dioxide from the reaction zone to maintain the partial pressure of oxygen in the system at a desired level.

U.S. Pat. No. 8,563,791 discloses a process of solubilizing organic solids by reacting organic solid with an oxidant in superheated water to form a solubilized organic solid. The oxidant is preferably pure, undiluted molecular oxygen. However, pure oxygen is not only costly, but can be dangerous. The process is performed in reactors with no headspace (a small accumulation of a flammable gas like methane or hydrogen (which will be released in a thermal cracking process) with oxygen in the headspace of a reactor can explode at higher temperatures of the process).

Jacobus J. Bergh et al., Non-catalytic oxidation of water-slurried coal with oxygen: identification of fulvic acids and acute toxicity Origin, 76 FUEL, 149-154 (1997) describes a process for aqueous oxidation of coal with oxygen to convert about 8% of coal to fulvic acids. They use a temperature of 180° C. and a pressure of 600 psig and a reaction time of 1 hour. They study the products for their toxicity as antibacterial agents.

In an earlier work, R. C. Smith et al., Oxidation of Carbonaceous Materials to Organic Acids by Oxygen at Elevated Pressures, 61 J. AM. CHEM. SOC., 2398-2402 (1939), describe alkali-oxygen oxidation of bituminous, high rank coal to produce a mixture of acids, as well as 50-60% CO₂. KOH was used at 6.8 times the weight of the coal and the temperature ranges from 100 to 250° C. and an oxygen pressure of 100 to 375 psig was applied.

One major drawback of the processes disclosed in the prior art is the use of relatively high temperature, pressure, and/or concentrations of solvents or oxidizing agents such as pure O₂ or other costly oxidizers. Such severe conditions result in prohibitive raw material or energy costs, making such processes uneconomical on an industrial scale. These processes also typically result in a product stream that is incompatible with a subsequent microbial conversion step.

An improved process is needed that utilizes milder conditions and yet employs efficient oxidative depolymerization of the carbonaceous materials and enhances the biodegradability of the resulting mixture to chemicals and biogas. Such an improved process can lower the cost of producing industrial raw materials from carbonaceous feedstocks thereby improving the economic viability of the process and its products.

In addition to vast resources of coal, one large source of carbonaceous materials which up to now appears to be underutilized is chemical pulping mills, as those, for example, used for production of paper and similar products.

Chemical pulping mills use a combination of basic reagents, heat and pressure, in an aqueous environment to dissolve and separate lignin and hemicellulose polymers of wood from cellulosic fibers. The cellulosic fibers are used to produce paper and paper-like products. The residual material containing degraded lignin, degraded hemicellulose, inorganics, and extractives (terpenes, tall oils, etc.), typically present in a caustic water solution, is generally termed “black liquor”. Black liquor is currently considered a waste product, with limited economic value.

Black liquor contains more than half of the energy content of the original wood entering the paper mill. Currently, the practice in the pulping mill industry is to concentrate the black liquor by dewatering it, and burning the concentrated black liquor in a recovery boiler to produce energy. Base reagents may also be recovered and recycled in the process.

Tall oils (liquid rosin) are typically removed from the black liquor prior to the concentration step as the solubility of tall oils decreases with dewatering. These tall oils are economically valuable products as they may be used as components in adhesives, emulsifiers, rubbers, inks, drilling fluids, diesel fuels (see, for example U.S. Pat. No. 8,471,081) or other products.

Even with the recovery of tall oil (which may contribute about 1 to 1.5% of the pulping mill's revenue), and energy generation by burning black liquor, the economic value of black liquor continues to be low. Various attempts have been made to produce more valuable products from the black liquor.

U.S. Pat. No. 4,436,586 discloses a method for producing both kraft pulp and alcohol from hardwood chips or the like. The wood chips are subjected to mild acid prehydrolysis following by mild caustic pre-extraction. The withdrawn hydrolysate has insufficient furfural to inhibit microorganism growth, and both the hexose and pentose sugars in the hydrolysate are fermented to ultimately produce ethanol, butanol, or the like. The chips, after caustic pre-extraction, are subjected to a sulphate cook, and a wash, and the resultant pulp is a kraft pulp said to have viscosity and tear strength characteristics more desirable than conventional kraft pulp. The pulp can be subjected to oxygen delignification, and a higher K number can be achieved in fewer subsequent bleaching stages than with conventional kraft pulp.

U.S. Pat. No. 8,445,563 discloses the utilization of kraft lignin in phenol or formaldehyde bonding resins for oriented strand boards (OSB's). According to this patent, the shelf-life and chemical emission properties in a liquid PF resin for use in OSB's can be improved by incorporation of a particular degraded lignin material that is isolated from black liquor generated in the kraft wood pulping process. Specifically, the degraded lignin material is incorporated into a liquid PF resin targeted for use in OSB's replacing some of the urea component, which results in a composition with the aforementioned advantages, as well as reduced raw material costs.

US 2012/0064609 discloses a method for contacting coal or lignocellulosic materials with a composition comprising a pyrophosphate or a derivative thereof. Solubilization of coal or lignocellulosic materials can be carried out in a subterranean formation, in a terrestrial formation or in an ex situ reactor. The method comprises the step of introducing a composition with a pyrophosphate or a derivative thereof into the coal or lignocellulosic materials so as to cause solubilization of the coal or lignocellulosic materials.

U.S. Pat. No. 2,193,337 discloses a process for producing oxalic acid salts by heating carbonaceous materials such as sawdust, woodchips, peat or coal, with oxygen-containing gases at elevated pressures and temperatures in the presence of at least 10 times the weight of carbonaceous material of water and preferably an oxide or hydroxide of an alkali or alkaline earth metal, in an amount of 1.5 to 4 times the weight of feedstock. The oxalic acid, as well as possibly other organic acids such as mellitic acid, benzoic acid, or acetic acid, may then be isolated from the resulting products. The examples in the patent show that a preferred temperature is 180° C., that the pressure should be maintained at 20 atmospheres and that a reaction time of 2 hours can be used.

Extraction of lignins from pulping processes is described in U.S. Pat. No. 4,764,596. After separation from the cellulosic pulps produced during the pulping process, the derivatives of native lignin are recovered from the black liquors by depressurization/flashing followed by dilution with cold water which will cause the fractionated derivatives of native lignin to precipitate thereby enabling their recovery by standard solid/liquid separation processes. Various disclosures exemplified by U.S. Pat. No. 7,465,791 and WO 2007/129921, describe modifications to this process for the purpose of increasing the yields of fractionated derivatives of native lignin recovered from fibrous biomass feedstocks during biorefining.

An improved process is needed that treats black liquor in a way to produce common small organic molecules that may be then used for further applications. Such a process is needed in order to improve the revenue for pulp mills, and to protect the environment by utilizing the black liquor more effectively.

### SUMMARY OF THE INVENTION

The present invention provides a method for treating a carbonaceous feedstock, comprising steps of oxidizing a mixture of a carbonaceous feedstock optionally with at least one solubilizing agent and water to a temperature below 300° C. and at a pressure below 1230 psig. One important feature of this invention is the fact that the carbonaceous feedstock gains mass from the insertion or addition of oxygen into the structure, resulting in the formation of oxygenated molecules and reduced amounts of CO₂ in comparison with known methods. This gain is considerable and can be more than 30% of the starting feedstock mass for carbonaceous materials in the liquid phase and more than 75% if CO₂ is included. The method may further comprise one or more subsequent separation steps and/or microbial digestion steps.

The present invention further provides a method for treating a carbonaceous feedstock using a combination of steam and air in a solid-vapor (non-aqueous) environment. These conditions can provide an advantage by ultimately raising the concentration of water soluble chemicals (lower water input and lower separation cost) in the final condensed product, lowering or even eliminating suspended solids (either incomplete reacted coal or ash minerals) from the resulting condensed product. Furthermore, the extent of reaction can be driven to the point of extinction of coal particles and generating ash as the only byproduct. The severity of conditions in terms of O₂/coal, steam/coal, vapor and solids residence times, and temperature can be varied to alter the product distribution and gain selectivity and yield to specific chemical products. This process does not require pure oxygen from any source (including air or peroxide), nor is pure oxygen desirable. Another major advantage of the present invention is the ability to operate at close to ambient pressure, which eliminates the cost of air compression, as well as reduces the cost of reactor equipment.

The methods of the present invention allow the production of various product distributions based on varying operating conditions of the process(es). For example, under certain conditions, a mixture of water soluble oxochemicals is produced such as aliphatic and aromatic carboxylic acids. In other conditions, a mixture of these oxochemicals and a mixture of waxy hydrocarbons containing paraffins and olefins ranging from C10 to C44 chain lengths are produced. These hydrocarbons are water insoluble and are easily separated from the aqueous phase as shown in the examples provided hereunder. In yet another aspect of the present invention, the fixed bed of coal in the configuration acts as a filter for coal particles, eliminating the need for separation of particulates from liquid products.

The present invention further provides a method for treating a black liquor feedstock, comprising a step of treating a black liquor in the presence of at least one oxidizing agent at a temperature below 300° C. and at a pressure below 1230 psig, to obtain one or more organic compounds.

### BRIEF DESCRIPTION OF THE DRAWINGS

FIG. 1 is a flow chart that shows a method according to one embodiment of the present invention.

FIG. 2 is a flow chart that shows an alternative method of the invention with a reaction product from the microbial digestion step being fed back to the heating step.

FIG. 3 is a schematic representation of a method according to another embodiment of the present invention.

FIG. 4 is a conceptual flow diagram for implementing a method according to one embodiment of the present invention.

FIG. 5 shows oxygenation of coal to make it more biodegradable by methods according to one embodiment of the present invention.

FIG. 6 shows oxygen retention efficiency in relation to starting O₂ in headspace, with or without CuO catalyst, by a method according to one embodiment of the present invention.

FIG. 7 shows degree of conversion of coal to dissolved carbon in a two-pass treatment of the coal, according to one embodiment of the present invention.

FIG. 8 shows the effect on bioavailability of oxidation via addition of air to alkali, according to one embodiment of the present invention.

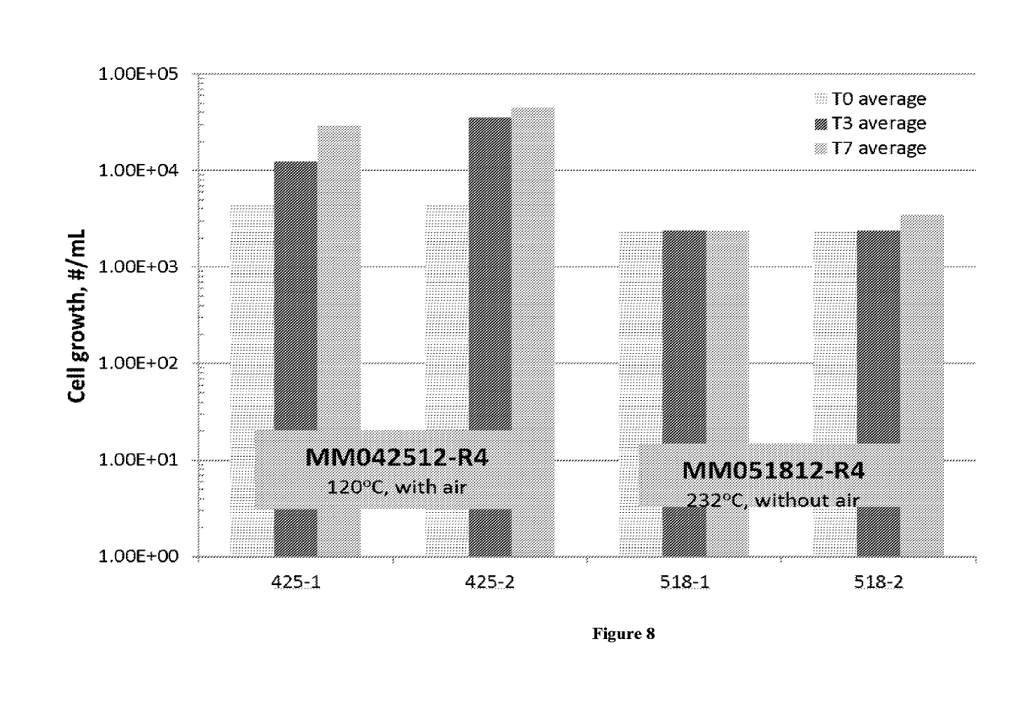

FIG. 9 is a flow chart that shows a prior art method of handling black liquor in a pulp mill.

FIG. 10 is a flow chart depicting a process according to one embodiment of the present invention.

FIG. 11 is a flow chart depicting a process according to another embodiment of the present invention, wherein selected organic polymers are recovered from the raw black liquor and only selected components of black liquor are used in to generate one or more organic compounds comprising from about 2 to about 20 carbon atoms.

FIG. 12 is a flow chart depicting a process according to another embodiment of the present invention, wherein only selected components of black liquor are used in to generate one or more organic compounds comprising from about 2 to about 20 carbon atoms, and the residue is treated further for energy recovery.

FIG. 13 is a flow chart depicting a process according to another embodiment of the present invention, wherein selected organic polymers are recovered from the raw black liquor, only selected components of black liquor are used in to generate one or more organic compounds comprising from about 2 to about 20 carbon atoms, and the residue is treated further for energy recovery.

FIG. 14 show a GCMS spectrum of an acid fraction of small organic compounds obtained by a method according to one embodiment of the present invention.

FIG. 15 shows a product distribution of small organic compounds obtained by a process in accordance with the present invention applied to a black liquor obtained from pine wood, in comparison to a product distribution for a product obtained from Powder River Basin (PRB) sub-bituminous coal.

FIG. 16 shows a simplified schematic of an aspect of the present invention showing a process for oxidative steam-stripping of coal as a carbonaceous feedstock.

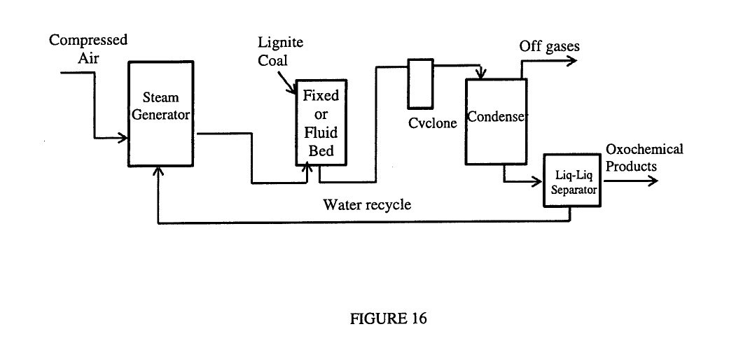

FIG. 17 shows formation of carboxylic acids from methods of the present invention (see Example 6) followed by pH and FTIR, indicating a maximum between 200-220 degrees C. based on the minimum pH and maximum intensity of the carboxylic peak in FTIR.

FIG. 18 shows an image of a 3-phase product mixture (showing a hydrocarbon waxy phase starting to appear in addition to the aqueous phase and an organic phase).

FIG. 19 shows a chromatogram resulting from GC-MS analysis of the waxy phase extracted by hexane.

FIG. 20 shows formation of carboxylic acids from methods of the present invention (see Example 7) followed by pH and FTIR (test performed at a relatively constant temperature of 200 degrees C.).

### DETAILED DESCRIPTION OF THE PREFERRED EMBODIMENT(S)

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

The term “substantially” means an amount of at least generally about 80%, alternatively about 90%, or alternatively about 99%.

As used herein, the term “carbonaceous feedstock” includes naturally occurring polymeric substances, such as coal, lignite, tar sands, tars, crude oils, peat, pitch, resins, lignin, latex rubber, waxes, agricultural wastes, bark, wood, any type of renewable biomass and other products from trees, algae cake, and other recalcitrant organic matter, and may also include lower-valued by-products from petroleum refining and chemical manufacturing, such as crude oil atmospheric bottoms, crude oil vacuum residues, residua from fluid catalytic cracking, petroleum coke, coker and other thermal cracking gas oils and bottoms, raffinates, asphalts, polynuclear aromatics, and the like, and may even include synthetic polymer wastes such as polyethylene, polypropylene, polystyrene, polyesters, polyacrylics, and the like.

In one embodiment of the present invention, the carbonaceous feedstock comprises coal, lignite, tar sands, tars, crude oils, peat, pitch, resins, lignin, latex rubber, waxes, petroleum coke, agricultural wastes, bark, wood, and algae concentrate.

Algae concentrate, such as algae paste or algae cake, is a residue to obtained by separating algae from the medium in which they grow, which is typically water based. The concentrated algae may be able to be processed in a form containing small amount of residual water. The algae may be separated from the medium in a variety of ways, for example, by filtration.

As used herein, the term “coal” refers to any of the series of carbonaceous fuels ranging from lignite to anthracite. The members of the series differ from each other in the relative amounts of moisture, volatile matter, and fixed carbon they contain. Coal is comprised mostly of carbon, hydrogen, sulfur, oxygen, nitrogen and entrained water, predominantly in the form of large molecules having numerous carbon double bonds. Low rank coal deposits are mostly comprised of coal and water. Coal is a mineral deposit containing combustible substances which is considered to be a fossil fuel. Coal is formed from plants that have been fossilized through successive deoxidation and condensation processes.

As used herein, the term “microorganism” includes bacteria, archaea and fungi. The microorganisms, by example, may include: Archaeoglobales, Thermotogales, Cytophaga group, Azospirillum group, Paracoccus subgroup, Sphingomonas group, Nitrosomonas group, Azoarcus group, Acidovorax subgroup, Oxalobacter group, Thiobacillus group, Xanthomonas group, Oceanospirillum group, Pseudomonas and relatives, Marinobacter hydrocarbonoclaticus group, Pseudoalteromonas group, Vibrio subgroup, Aeromonas group, Desulfovibrio group, Desulfuromonas group, Desulfobulbus assemblage, Campylobacter group, Acidimicrobium group, Frankia subgroup, Arthrobacter and relatives, Nocardiodes subgroup, Thermoanaerobacter and relatives, Bacillus megaterium group, Carnobacterium group, Clostridium and relatives, and archaea such as Methanobacteriales, Methanomicrobacteria and relatives, Methanopyrales, and Methanococcales.

More specific examples of microorganisms may include, for example, Aerobacter, Aeromonas, Alcaligenes, Bacillus, Bacteroides, Clostridium, Escherichia, Klebsiella, Leptospira, Micrococcus, Neisseria, Paracolobacterium, Proteus, Pseudomonas, Rhodopseudomonas, Sarcina, Serratia, Streptococcus and Streptomyces, Methanobacterium omelianskii, Mb. Formicium, Mb. Sohngenii, Methanosarcina barkeri, Ms. Methanica, Mc. Masei, Methanobacterium thermoautotrophicum, Methanobacterium bryantii, Methanobrevibacter smithii, Methanobrevibacter arboriphilus, Methanobrevibacter ruminantium, Methanospirillum hungatei, Methanococcus vannielli, Methanothrix soehngenii, Methanothrix sp., Methanosarcina mazei, Methanosarcina thermophila, Methanobacteriaceae, Methanosarcinaceae, Methanosaetaceae, Methanocorpusculaceae, Methaanomicrobiaceae, other archaea and any combination of these.

As used herein, the term “microorganism consortium” refers to a microorganism assemblage, containing two or more species or strains of microorganisms, and especially one in which each species or strain benefits from interaction with the other(s).

As used herein, the term “bioconversion” refers to the conversion of carbonaceous materials into a product that may include methane and other useful gases and liquid components by a microorganism. The product of bioconversion includes, but is not limited to, organic materials such as hydrocarbons, for example, methane, ethane, propane, butane, and other small organic compounds, as well as fatty acids and alcohols, that are useful as fuels or chemicals or in the production of fuels or chemicals, and inorganic materials, such as gases, including hydrogen and carbon dioxide.

The present invention provides a method of converting at least part of a carbonaceous feedstock to converted products and biodegradable substrates. The invention can simultaneously oxidize, depolymerize, reform and/or solubilize low-valued high molecular weight carbonaceous materials in the carbonaceous feedstock to lower molecular weight hydrocarbons, oxo-chemicals and other chemicals. Here, oxo-chemicals are organic compounds that comprise at least one oxygen atom.

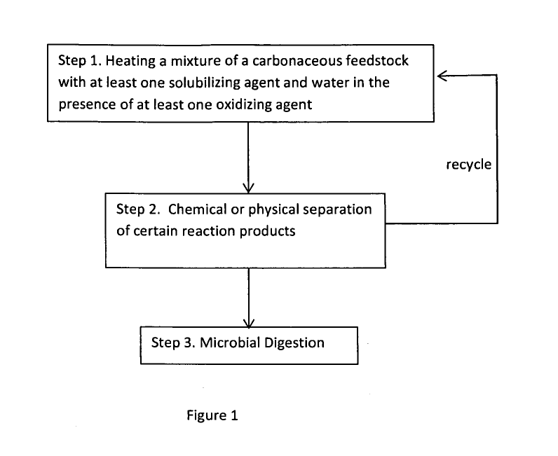

Referring to FIG. 1, the present invention includes a step of heating a mixture of a carbonaceous feedstock optionally in the presence of at least one solubilizing agent and water in the presence of at least one oxidizing agent. The heating step may comprise raising the temperature of the mixture to a desired temperature and/or keeping the mixture at a pressure at or above the steam saturation pressure. In some embodiments, the reaction product may optionally be subjected to chemical and/or physical separation and/or microbial digestion.

Chemical and/or physical separation may be employed for separation of various components in the reaction product. For example, some high-valued minerals and chemicals may be retrieved from the reaction product using conventional chemical and/or physical separation methods. Such chemicals include, for example, oxo-chemicals. Applicable chemical and physical separation technologies that may be used include any of those known to one skilled in the art, including fractional distillation, liquid/liquid extraction, reactive extraction, electrodialysis, adsorption, chromatography, ion exchange, membrane filtering, and hybrid systems.

In some embodiments, the carbonaceous feedstock may be too impermeable, e.g. due to their limited porosity, to be efficiently treated by the heating step. In such a case, the carbonaceous feedstock may be preprocessed (e.g. comminuted) to increase its permeability or available surface area, thus increasing the susceptibility of the large carbonaceous molecules in the carbonaceous feedstock to the treatment of the present invention. Any method known to a skilled person in the art that is suitable for reducing the particle size of carbonaceous feedstocks may be used for the present invention. For example, physical (e.g., grinding, milling, fracture and the like) and chemical approaches (e.g., treating with surfactants, acids, bases, oxidants, such as but not limited to acetic acid, sodium hydroxide, percarbonate, peroxide and the like) can be applied to reduce the size of the carbonaceous materials in the carbonaceous feedstock. In some embodiments, preprocessing may be used to break down coal, oil shale, lignite, coal derivatives and like structures to release more organic matter, or to make them more vulnerable to degradation into smaller organic compounds. Some suitable preprocessing methods are described in U.S. Patent Application Publication No. 2010/0139913, International Patent Publication No. WO 2010/1071533 and U.S. Patent Application Publication No. 2010/0262987, the disclosures of which are hereby incorporated by reference herein.

In one embodiment, coal and water at about a 1:2 weight ratio are loaded into a mill with steel media. The duration of milling may be in the range from 60 to 90 minutes. After milling, the coal slurry may be used as an input to the heating step of the process of the present invention.

The solubilizing agent that can be optionally used in the present invention may be selected from mineral acids or mineral bases. Preferred bases include Group I (alkali metals) and Group II (alkaline earth) oxides, hydroxides, carbonates, borates, or halogenates. In particular, sodium, potassium, calcium, and magnesium compounds are preferred. Examples of the solubilizing agents include sodium hydroxide, potassium hydroxide, ammonium hydroxide, sodium carbonate, sodium bicarbonate and potassium carbonate, or any mixture of these. Naturally occurring minerals of some of these materials are also appropriate for use in this process. These include, but are not limited to Nahcolite, Trona, Thermonatrite, Gaylussite, Hydromagnesite, Lansfordite, Ikaite, Hydrocalcite, Dolomite, Huntite, Aragonite, Natrite, Magnesite, Calcite, Kalcinite, Gregoryite, and others.

The mineral bases generally comprise no more than 15 wt % of the mixture provided to the heating step, and preferably comprise below 10 wt % and most preferably at or below 6 wt % of the mixture provided to the heating step. In some embodiments, the solubilizing agent comprises at least 1 wt % or at least 3 wt % or at least 5 wt % of the mixture fed to the heating step.

In some embodiments, the solubilizing agent may be a mineral acid, such as phosphoric acid, nitric acid, boric acid, hydrochloric acid, and sulfuric acid.

The carbonaceous feedstock may be mixed with the solubilizing agent provided in an aqueous solution to make the mixture. In some alternative embodiments, the carbonaceous feedstock may be combined with steam or water vapor containing solubilizing agent. In these embodiments, the vapor or steam may be blown onto the carbonaceous feedstock.

In some embodiments, the carbonaceous feedstock is dispersed in an aqueous solution of the solubilizing agent to make the mixture. The amount of carbonaceous feedstock dispersed in water is limited by the average size of the monomer molecules that may be oxidatively reformed from the carbonaceous feedstock and their solubility in water based on their functional groups, the degree of ionization they have in water, and physical and chemical attributes of the aqueous system, such as temperature, pH, pressure, activity coefficient, and other considerations. Solution viscosity also increases with higher carbonaceous feedstock loading in the slurry-like mixture and is a limitation that may reduce mass transfer and mixing between the solid and liquid. In some embodiments, the carbonaceous feedstock content in the mixture may be less than 40% by weight. The carbonaceous feedstock content of the mixture may be at or below 30% by weight or at or below 25% by weight.

In some embodiments, at least one catalyst may optionally be added to the mixture. The catalyst may catalyze the oxidation reaction by, for example, causing or enhancing formation of peroxides and superoxides, which may enhance the rate of oxygen insertion into the carbonaceous material relative to complete oxidation of the carbonaceous material.

The catalyst may be selected from water insoluble metals, transition metals, and precious metals, or their salts or oxides. Examples of these metals include nickel, cobalt, platinum, palladium, rhenium, copper, iron, zinc, vanadium, zirconium and ruthenium. The catalyst may be unsupported or may be supported on inert or active matrix material such as clay, alumina, silica, silica alumina, zeolites, activated carbon, diatomaceous earth, titania, zirconia, molybdena, ceramics, and the like. Such catalysts can enhance rates of oxygen transfer, insertion and reforming of high molecular weight carbonaceous compounds as well as being able to enhance the degree of relative oxidation. Examples of the catalysts include metal oxides, mixed metal oxides, hydroxides, and carbonates, of cerium, lanthanum, mixed rare earths, brucite, hydrotalcite, iron, clays, copper, tin, and vanadium.

In some embodiments, the catalyst used in the present invention is a solid catalyst containing activated carbon. The type of activated carbon suitable for use as a catalyst in the present invention is not specifically limited. Suitable activated carbons may be selected from materials such as charcoal, coal, coke, peat, lignite and pitch. Suitable activated carbons also include carbon fibers, such as activated carbon fibers of the acrylonitrile family, the phenol family, the cellulose family, and the pitch family.

Activated carbon has a property of absorbing oxidizable substances from the carbonaceous material onto its surface. The adsorption of oxidizable substances onto the catalyst surface creates chemical bonding, altering the electron density around the molecules of the oxidizable substance and allowing the molecules to undergo oxidation with higher efficiency. For the purpose of catalyzing the oxidation reactions, the type and amount of polar groups on the surface of the activated carbon can change the properties of activated carbon. The amount or type of polar groups on the surface of the activated carbon affects the formation of chemical bonds with oxidizable substances. Thus, the performance of the activated carbon as a catalyst changes considerably in accordance with the amount and type of polar groups introduced into the catalyst. If the oxidizable substances are mostly organic substances and/or inorganic anionic substances, the activated carbon catalyst may contain a small amount of polar groups, which give the catalyst hydrophobic properties for more efficient catalysis of oxidation. The activated carbon catalysts suitable for oxidizing large organic substances are described in more details in European patent No. EP 1116694 B1, which is incorporated herein by reference.

The amount of polar groups on the surface of activated carbon may be controlled by varying the process of producing the activated carbon catalyst. For example, U.S. Pat. No. 3,996,161 describes a method of preparing active carbon for treatment of waste liquid comprising immersing powdered coal in an aqueous solution of a polar compound containing a non-polar group bonded to a polar group, and then washing the immersed coal followed by drying of said washed coal. This document is incorporated by reference in its entirety herein. By varying the polar compound or its amount in the aqueous solution, activated carbon with different levels of polar groups may be produced.

In some embodiments, the carbonaceous material itself, especially the large carbonaceous molecules and resident mineral and associated ions, can function as a catalyst to catalyze the oxidative disruption or depolymerization of the carbonaceous material. In these embodiments, the interaction among the large carbonaceous molecules on the surface of the carbonaceous material may engage in chemical bonding or alter the electron density around the large carbonaceous molecules, which can facilitate oxidation and depolymerization of the large carbonaceous molecules in the carbonaceous material. In one embodiment, the carbonaceous material is coal and the coal itself functions as a catalyst for oxidation and depolymerization of the coal.

The mixture containing the carbonaceous material is heated in a reaction vessel in the presence of at least one oxidizing agent. The heating step may comprise raising the temperature of the mixture to a desired temperature by any suitable means and/or subjecting the mixture to a pressure at or above the steam saturation pressure. Multiple reactions may occur during the heating step, including oxidation, depolymerization, reforming and solubilization. In a reforming process, the molecular structure of a hydrocarbon is rearranged.

The oxidizing agent may be selected from air, oxygen enriched air, oxygen, ozone, sulfuric acid, permanganates, carbon dioxide, nitrous oxide, nitric acid, chromates, perchiorates, persulfates, superoxides, chlorates, peroxides, hypochlorites, Fenton's reagent and nitrates in which the cations may comprise metal cations, hydrogen ions and/or ammonium ions.

Oxidizing agents may be ranked by their strength. See Holleman et al. “Inorganic Chemistry,” Academic Press, 2001, page 208. A skilled person will appreciate that, to prevent over-oxidation of the carbonaceous materials, the conditions in the heating step may be adjusted according to the strength of the oxidizing agent used. For example, when a strong oxidizing agent is used, one or more of temperature, pressure, and duration of the heating step may be reduced to prevent over-oxidation and/or ensure that the desired degree of conversion is not exceeded. On the other hand, when a weak oxidizing agent is used, one or more of temperature, pressure, and duration of the heating step may be increased to ensure that the desired degree of oxidation and/or conversion is achieved. When the oxidizing agent is gaseous, the pressure in the reaction vessel for the heating step is important for ensuring the desired degree of oxidation and/or conversion.

In some embodiments, oxygen is used as the oxidizing agent. In one embodiment, oxygen can be delivered to the reaction vessel as air. In some other embodiments, depending on the susceptibility of the carbonaceous feedstock to oxidation, oxygen-enriched air can be used. Suitable enrichment percentages can be from an oxygen concentration slightly above that of atmospheric air to substantially pure oxygen.

One important feature of the present invention is a considerable mass gain of the feedstock due to added or inserted oxygen in the carbonaceous material. This applies to both liquid and solid feedstock and has a significant positive impact on the economics of the process. In addition, the gain in bioavailability resulting from the incorporation of oxygen into the polymeric carbonaceous molecules in the feedstock and its subsequent breakdown is very beneficial. In fact, even the residual coal solids (partially converted, partially oxidized) are more oxygenated at the surface and this makes them more bioavailable as a soil nutrient, as well.

The reaction vessel in which the heating step is conducted is not limited to any particular reactor design, but may be any sealable multiphase reaction vessel that can tolerate the temperature and pressure required for the present invention. In some embodiments, the mixture is fed to a reactor, which has been pre-heated to the desired temperature. Then, air or oxygen enriched air is slowly added to the reactor until the desired pressure is reached. The temperature and pressure in the reactor may be monitored during the filling of air or oxygen enriched air, as well as during the heating step itself. Some reactor design is described in Blume (“Bitumen blowing unit converts residues to asphalt,” Hydrocarbon Processing, March 2014), which is incorporated herein by reference.

The mixture in the reaction vessel is heated to a temperature below 300° C. (572° F.), or below 220° C. (428° F.), or below 150° C. (302° F.). A positive pressure in the reaction vessel is maintained at saturated steam pressure or slightly higher, for example below 1230 psig, or below 322 psig, or below 54 psig respectively. A minimum temperature is approximately 130° C. and a respective minimum pressure is approximately 24 psig.

The mixture in the reaction vessel has at least two phases: a liquid phase (water/solubilizing agent/oxidizing agent) and a solid phase (carbonaceous feedstock). In many embodiments, there are three phases in the reaction vessel: gas (oxygen/air and/or steam), liquid (water/solubilizing agent) and solid (carbonaceous feedstock). To ensure efficient heat and mass transfer among these phases, the mixture may be subjected to mechanical or other means of agitation. The reaction vessel may include structural features to facilitate interactions among the phases. For example, an unstirred reaction vessel with gas dispersion features, a reaction vessel with mechanical agitation devices as well as reaction vessels with gas entrainment devices or combinations thereof. Exemplary reactors include a co-current flow tubular reactor with gas dispersion, a counter-current flow tubular reactor with gas dispersion, and a flowing tubular reactor with static mixers.

In some embodiments, the reaction vessel is a bubble column reactor configured to enhance mass transfer of oxygen from the gas phase to the liquid and solid phases. The bubble column reactor typically consists of vertically arranged cylindrical columns. Bubble columns are configured such that gas, in the form of bubbles, rises in the liquid or slurry phase in contact with the liquid and dispersed solids. The introduction of gas to the reactor takes place at the bottom of the column and causes a turbulent stream to enable an optimum oxygen transfer to the liquid phase as the bubbles raise to the top surface of the liquid phase. The interaction between the gas, liquid and solid phases is enhanced with much less energy than would be required for mechanical stirring. The liquid phase can be in parallel flow or counter-current flow with the gas phase. The gas, escaping from the top surface of the liquid phase may be recycled back to the bubble column reactor and reintroduced back to the bottom of column. The vessel may also have a conical shape with progressive increase in diameter at the bottom to increase the solids residence time for a more efficient conversion.

The bubble column reactor can facilitate chemical reactions in a multi-phase reaction medium because agitation of the reaction medium is provided primarily by the upward movement of gas bubbles through the reaction medium. The diameter of the bubbles can be correlated with the efficiency of gas-liquid mass transfer, since the bubble size has a strong influence on hydrodynamic parameters such as bubble rise velocity, gas residence time, gas-liquid interfacial area and the gas-liquid mass transfer coefficient. A person skilled in the art may determine the optimal size or size distribution of the bubbles¹ for achieving efficient oxidiation/depolymerization of the carbonaceous material (Kantarci et al., “Bubble column reactors,” Process Biochemistry, vol. 40, pages 2263-2283 (2005)). Because different types of carbonaceous materials have very diverse characteristics, the size of the bubbles may be adjusted depending on the characteristics of the carbonaceous material and the desired pretreatment products

In some other embodiments, the reaction vessel is a trickle bed reactor configured to enhance mass transfer of oxygen from the gas phase to the liquid phase. In a trickle bed reactor, the liquid phase and gas phase flow concurrently downward through a fixed bed of catalyst particles on which reaction takes place. At sufficiently low liquid and gas flow rates, the liquid trickles over the catalyst packing in essentially a laminar film or in rivulets, and the gas flows continuously through the voids in the bed. This is sometimes termed the gas continuous region or homogeneous flow, which enhances oxygen transfer from the gas phase to the liquid phase. Trickle bed reactors have complicated and as yet poorly defined fluid dynamic characteristics. Contact between the catalyst and the dispersed liquid film and the film's resistance to gas transport into the catalyst, particularly with vapor generation within the catalyst, is not a simple function of liquid and gas velocities. The maximum contact efficiency is attainable with high liquid mass velocities, e.g. 1-8 kg/m², or 2-5 kg/m². A detailed description of trickle bed reactors and other multiphase reactors can be found under the heading “Reactor Technology” in “Kirk-Othmer Encyclopedia of Chemical Technology”, Third Edition, Volume 19, at pages 880 to 914, which is hereby incorporated herein by reference.

Trickle bed reactors may be operated in various flow regimes, depending on vapor and liquid flow rates and properties. It should be noted, however, that the operating window of trickle flow is very wide and not only determined by flow rates (see, e.g., E. Talmor, AlChE Journal, vol. 23, pages 868-874, 1977, which is hereby incorporated herein by reference). Thus, for instance, it may be possible to operate the trickle bed reactor with low liquid flow rates in conjunction with relatively high gas rates in some embodiments.

The duration of the heating step may be determined, for example, by the oxidative stress induced in the mixture and the desired product. As a general rule, a higher oxidative stress requires a shorter duration heating step. In addition, if the desired products are generated by more complete oxidation of the carbonaceous materials, e.g. via a series of sequential reaction steps, a longer duration heating step may be required.

Reaction times can vary from a few seconds to several hours, depending on the degree of conversion required, the reduction in molecular weight desired, the reactivity of the feedstock, process economics, the amount of carbon dioxide, carbon monoxide, and hydrogen generated, and other constraints. In one embodiment, the carbonaceous feedstock is coal and the reaction time is in the range from about 0.5 to about 4 hours, or about 1 to about 3 hours, or about 2 hours.

In some embodiments, the reaction conditions including temperature, pressure and reaction time may also depend on molecular and elemental characteristics of the particular carbonaceous feedstock. Examples of the characteristics of the carbonaceous feedstock which may be taken into consideration are the degree of aromaticity, the hydrogen to carbon ratio, oxygen to carbon ratio, nitrogen to carbon ratio, sulfur to carbon ratio, mineral or ash content, and other factors. Thus, in some embodiments, a blend of carbonaceous feedstocks of different characteristics may enhance the efficiency of the method by adjusting one or more of these characteristics. For example, blending a highly aromatic, more difficult to react, carbonaceous material, such as coal, with a more acyclic carbonaceous material, such as agricultural waste or synthetic polymer waste, will result in an oxidized product stream that is more biodegradable and will support greater microbial population densities, as well as increase the rate and depth of conversion of the less reactive molecules. The blending of feedstock technique is described in US 2012/0160658, incorporated herein by reference.

The extent of conversion can be controlled by using different reaction conditions to yield different types and amounts of, for example, partial oxidation products. The reaction conditions may also be adjusted to eliminate converted coal solids, other than inorganics concentrated in an ash stream, without significant loss of carbonaceous compounds to CO₂ production.

In some embodiments, a portion of the gaseous phase in the reaction vessel may optionally be continuously or periodically withdrawn and replaced. Carbon dioxide formed during the reaction has several roles, including acting as an excess base neutralizer and forming a carbonate buffering system in the water. A carbonate buffered system is a desirable feature for enhancing the subsequent microbial conversion to gas and chemicals. In many cases, microbes of interest prefer a system at or around pH 7. The CO₂ produced in the process reacts with excess base and reduces or eliminates the need to adjust the pH of the product stream resulting from depolymerization by the addition of acid, thereby lowering costs. The CO₂ also retains some of the mineralized carbon in the system, some of which can be reduced by certain microbes to beneficial products during their overall metabolism of oxidized carbonaceous materials. Any excess carbon dioxide formed during the reaction is preferably removed from the reaction vessel. In one embodiment, gas is withdrawn from the reaction vessel, the carbon dioxide content of the withdrawn gas is reduced and the gas with the reduced carbon dioxide content is optionally resupplied back to the reaction vessel, with or without being enriched with oxygen. This embodiment may be used for maintaining a desired partial pressure of oxygen in the reaction vessel during the reaction.

Some of the carbonaceous material in the feedstock may be oxidized to carbon dioxide and be subsequently converted to an alkaline carbonate. Therefore, it may be desirable to use a sufficiently alkaline solution to fix some, most or all of the carbon dioxide generated by the conversion reaction to maintain a higher level of partial pressure of oxygen when the oxidizing agent is oxygen or oxygen-enriched air. Otherwise, the formation of carbon dioxide in the reaction may reduce the partial pressure of oxygen in the system to a point where the conversion reaction will slow down and eventually cease.

In some embodiments, samples of the gas phase in the reaction vessel may be taken periodically in order to monitor the progress of the reaction. The gas sample may be analyzed by, for example, a gas chromatograph to identify the content of one or more components to provide an indication of the progress of the reaction. Once the desired degree of conversion is reached, the heating step may be terminated. Carbon dioxide may be withdrawn or oxygen may be periodically or continuously added to the reaction vessel for maintain the desired level of oxidant.

The method of the present invention can be conducted in batch, semi-batch, or continuously. In one aspect, the present invention oxidizes the carbonaceous material in the carbonaceous feedstock. At least portion of the carbonaceous material may be oxidized to organic acids, such as oxalic acid, mellitic acid, benzoic acid and acetic acid. In addition, high molecular weight carbonaceous compounds may be depolymerized/reformed to lower molecular weight carbonaceous compounds. In some embodiments, mineral bases are used to increase the pH of the mixture to a caustic alkaline pH of greater than 7, greater than 9 or greater than 10. In such mixtures, the formed organic acids will be present in salt form due to the presence of the mineral base. Such salts may be recovered from the reaction products by filtering off the solid material and extracting the oxalic acid therefrom with dilute hydrochloric or sulfuric acid. The salts of mellitic acid and like acids can be isolated from the filtrate by acidifying, warming, and filtering the warm liquid, while acetic acid can be recovered from the residual liquid by, for example, steam distillation.

The products of the reaction vessel may include minerals, chemicals and low-molecular weight carbonaceous compounds. These products may be used as raw materials for various industries such as the chemical, polymer, textile, and pharmaceutical industries. Metals may be recovered from the reaction product. The solids in the reaction product may also have value as fertilizer, fillers for cement and asphalt, and other such materials.

After extracting the minerals and high-value chemicals, the remainder of the reaction product may be subjected to microbial digestion. This portion of the reaction product includes solubilized carbonaceous compounds, and possibly some solid high molecular weight carbonaceous materials. Both fractions have gained considerable bioavailability from the oxidative pretreatment as a direct result of the incorporation of oxygen into the polymeric carbonaceous molecules in the feedstock and its subsequent breakdown. These products may be introduced to a microbial digester, where the carbonaceous materials, especially the low-molecular weight carbonaceous materials produced by oxidation and depolymerization, undergo a bioconversion process. During the bioconversion process, some, or all, of the carbonaceous materials are digested by the microorganism in the microbial digester. In one embodiment, the bioconversion process may produce biogases such as methane, hydrogen, carbon monoxide, other gases and mixtures thereof, which may be used as fuel or can be converted to electricity.

The conditions in the microbial digester should be optimized to achieve the greatest biodegradation of the carbonaceous materials in the digester, including one or both of the degree and rate of bioconversion. The reaction products obtained from the heating step may affect one or both of the degree and rate of bioconversion in a subsequent bioconversion. Thus, in one aspect of the invention, the conditions of the heating step are selected on the basis of producing reaction products that may include larger quantities of biodegradable materials and/or may exhibit an enhanced rate of biodegradation or an enhanced tendency to biodegrade.

The microbial digester may be either an aerobic digester or an anaerobic digester, or a combination of the two. In an aerobic digester, oxygen is supplied to the digester, which generally leads to fast breakdown of the carbonaceous materials fed into the digester. In an anaerobic digester, no oxygen is supplied to the digester. The breakdown of the carbonaceous materials in an anaerobic digester is generally slower. In some embodiments, both aerobic and anaerobic digesters may be used. Aerobic digestion and anaerobic digestion typically provide different products. Thus, aerobic and anaerobic digestion may function complimentarily.

In some embodiments, the microbial digester may be a partial anaerobic digester, which may be configured such that only portion of the microbial digester is exposed to oxygen. At another portion of the microbial digester, the oxygen has been essentially consumed and thus this portion of the microbial digester functions as an anaerobic digester. In this partial anaerobic digester, the carbonaceous materials pass from the aerobic portion to anaerobic portion of the microbial digester such that the carbonaceous materials are subjected to both aerobic digestion and anaerobic digestion. In some embodiments, the microbial digester may be supplied with limited oxygen. After the initial aerobic digestion, the oxygen is essentially consumed. Then the digester becomes an anaerobic digester.

The carbonaceous materials in the microbial digester are metabolized using microbes in the form of a single species or strain of a microorganism, multiple species or strains of microorganism or a microorganism consortium, in order to reduce carbonaceous materials, such as low molecular weight carbonaceous compounds, to other products of interest, including gases such as methane and hydrogen, liquids such as organic acids and alcohols, and solids such as oxo-aromatics.

Different microorganisms may be employed for different purposes. For example, two or more different reactions may be carried out in a single microbial digester by introduction of different microorganisms. Concentrations of microorganisms may also be varied to alter the relative reaction rates thereby influencing the reaction product mixture, particular in situations where reactions compete for the same reactants. A particular microorganism that is involved in a rate-limiting step of the bioconversion process may be supplemented to increase the reaction rate or yield of that rate-limiting step.

In embodiments employing a microorganism consortium, different species of microorganisms may be provided for different purposes. For example, a particular microorganism can be introduced for the purpose of increasing a nutrient, decreasing a concentration of a toxin, and/or inhibiting a competing microorganism for another microorganism in the consortium that participates in the conversion process. One or more species of microorganisms may be introduced to accomplish two or more of these purposes.

The microorganisms may be naturally occurring or may be synthesized from naturally occurring strains. Furthermore, the microorganisms may incorporate genetically modified organisms. These microorganisms may include fungi, bacteria, archaea, and combinations thereof. The microorganisms are typically selected to based on metabolic pathways that achieve conversion of carbonaceous molecules to specific products of interest.

In some embodiments, at least one nutrient may be introduced to the microbial digester. The nutrients may be substances upon which one or more species of microorganism is dependent or the nutrients may substances that can or will be converted to a substance upon which one or more species of microorganism is dependent. Suitable nutrients for the present invention include ammonium, ascorbic acid, biotin, calcium, calcium pantothenate, chlorine, cobalt, copper, folic acid, iron, K₂HPO₄, KNO₃, magnesium, manganese, molybdenum, Na₂HPO₄, NaNO₃, NH₄Cl, NH₄NO₃, nickel, nicotinic acid, p-aminobenzoic acid, biotin, lipoic acid, mercaptoethanesulfonic acid, nicotinic acid, phosphorus, potassium, pyridoxine HCl, riboflavin, selenium, sodium, thiamine, thioctic acid, tungsten, vitamin B6, vitamin B2, vitamin B1, vitamin B12, vitamin K, yeast extract, zinc and mixtures of one or more of these nutrients.

In some embodiments, at least one enzyme may also be added to the microbial digester. The enzymes can be used, for example, to enhance the conversion of carbonaceous materials. For example, an enzyme may be used to assist a specific conversion reaction, preferably a rate limiting reaction, in the bioconversion process. In some exemplary embodiments, enzymes may be used to further to enhance the yield, rate and/or selectivity of the bioconversion process, or a substance that inhibits growth of at least one species inhibitory to the yield, rate and/or selectivity of the conversion process.

The enzymes that are suitable for the present invention may include Acetyl xylan esterase, Alcohol oxidases, Allophanate hydrolase, Alpha amylase, Alpha mannosidase, Alpha-L-arabinofuranosidase, Alpha-L-rhamnosidases, Ammoniamonooxygenase, Amylases, Amylo-alpha-1,6-lucosidase, Arylesterase, Bacterial alpha-L-rhamnosidase, Bacterial pullanases, Beta-galactosidase, Beta-glucosidase, Carboxylases, Carboxylesterase, Carboxymuconolactone decarboxylase, Catalases, Catechol dioxygenase, Cellulases, Chitobiase/beta-hexo-aminidase, CO dehydrogenase, CoA ligase, Dexarboxylases, Dienelactone hydrolase, Dioxygenases, Dismutases, Dopa 4,5-dioxygenase, Esterases, Family 4 glycosylhydrolases, Glucanaeses, Glucodextranases, Glucosidases, Glutathione S-transferase, Glycosyl hydrolases, Hyaluronidases, Hydratases/decarboxylases, Hydrogenases, Hydrolases, Isoamylases, Laccases, Levansucrases/Invertases, Mandelate racemases, Mannosyl oligosaccharide glucosidases, Melibiases, Methanomicrobialesopterin S-methyltransferases, Methenyl tetrahydro-methanopterin cyclohydrolases, Methyl-coenzyme M reductase, Methylmuconolactone methyl-isomerase, Monooxygenases, Muconolactone delta-isomerase, Nitrogenases, O-methyltransferases, Oxidases, Oxidoreductases, Oxygenases, Pectinesterases, Periplasmic pectate lyase, Peroxidases, Phenol hydroxylase, Phenol oxidases, Phenolic acid decarboxylase, Phytanoyl-CoA dioxygenase, Polysaccharide deacetylase, Pullanases, Reductases, Tetrahydromethan-opterin S-methyltransferase, Thermotoga glucanotransferase and Tryptophan 2,3-dioxygenase.

In some embodiments, carbon dioxide, carbon monoxide, and hydrogen produced in the heating step may also be fed to the microbial digester, where specific microorganisms can convert these to small organic acids, hydrogen, alcohols, methane, carbon monoxide, carbon dioxide, and combinations thereof.

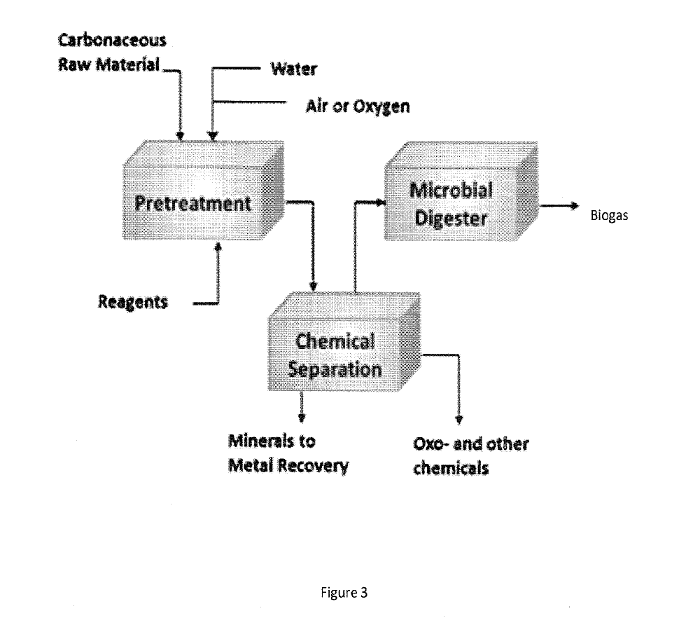

A schematic representation of the method according to one embodiment of the present invention is depicted in FIG. 3. The carbonaceous feedstock raw material is mixed with reagents, water and air or oxygen-enriched air in a pretreatment process. The reagents include at least one solubilizing agent, at least one oxidizing agent, and optionally a catalyst. The pretreatment process also includes heating the mixture to a suitable temperature and a suitable pressure.

The reaction product from the heating step (pretreatment) then undergoes chemical separation where minerals, oxo-chemicals and other chemicals are separated from the reaction product. The remainder of the reaction product is introduced into a microbial digester for bioconversion to produce biogas.

There are two significant purposes for the pretreatment step, enhancing biodegradability in the microbial digester and converting the carbonaceous material to minerals and desired chemicals. In some embodiments, it may be desirable to conduct the heating step as multiple sequential steps in order to better achieve both purposes to satisfaction. For example, if a first heating has its conditions optimized for higher biodegradability, complete oxidative cracking solubilization of the carbonaceous feedstock may not be achieved. The present invention thus encompasses methods where two or more sequential heating steps are conducted under different conditions.

In some embodiments, two or more sequential heating steps may be conducted under different conditions using the reaction product of a previous step as the feed to the following step. The reaction conditions at each sub-step are adjusted to favor different reactions, rates of reaction, degrees of conversion, etc. The reaction product from one sub-step or one or more components thereof may be fed to the next sub-step. For example, one sub-step may have reaction conditions selected for the production of valuable oxo-chemicals and another sub-step may have its reaction conditions selected for enhancing biodegradability of the reaction products.

Alternatively, the reaction product may be altered in some way before feeding it to the following step by, for example, chemically or physically separating one or more components of the reaction product. Also, the reaction product or one or more components thereof may be recycled to the initial heating step. At least one additional pass through the heating step can be used to enhance or complete conversion and solubilization of the carbonaceous materials in the carbonaceous feedstock. An example of a component of products to be recycled is partially converted solids which can be separated by mechanical means. Filtering, settling, centrifuging, hydrocycloning and other techniques may be used to separate unconverted or partially converted larger particulate product materials from the solubilized carbonaceous materials. These larger, typically partially oxidized (reacted) materials may then be further reacted to smaller materials by being recycled due to the longer combined residence time achieved via the recycle step.

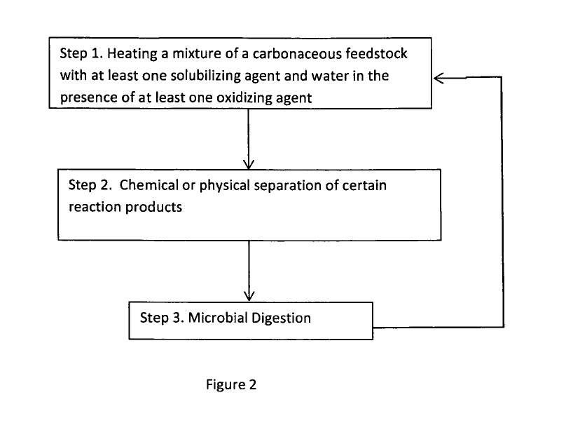

Referring to FIGS. 1-2, in some embodiments, the method of the present invention may be configured to recycle the reaction product or a component thereof from the heating, microbial digestion and/or chemical or physical separation steps back to the heating or communition step. Optionally, the reaction conditions at the later pass of the heating step may be different from the reaction conditions of the first pass through the heating step.

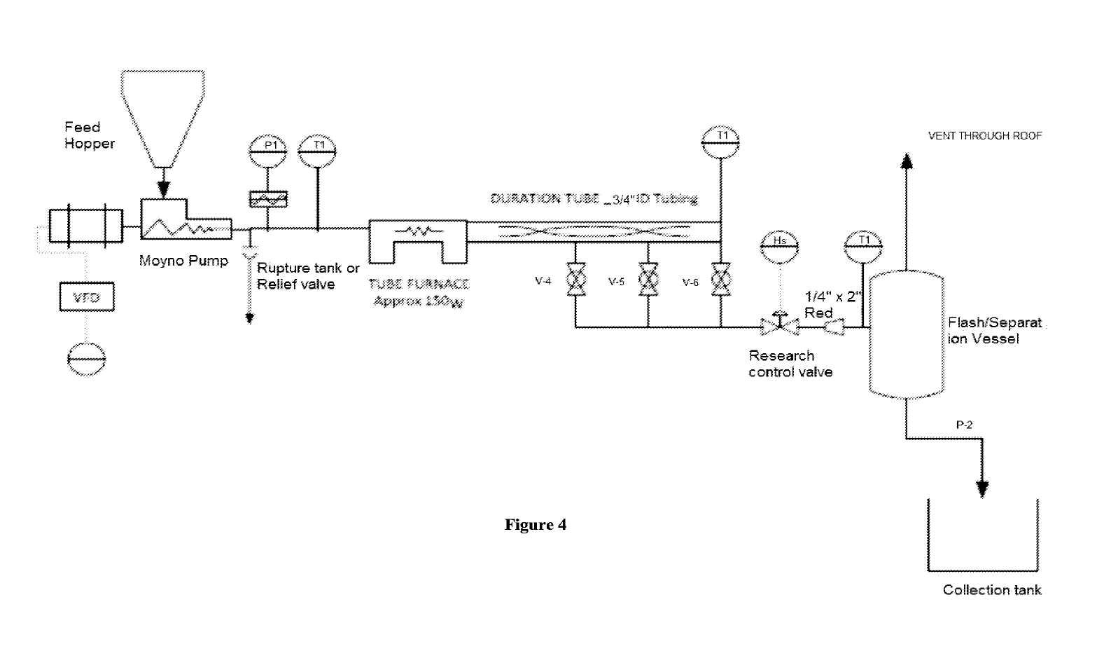

Referring to FIG. 4, the method of the present invention may be configured as recycling materials from microbial digester containing metallic ions and unconverted carbonaceous material back to the step (2) to enhance the efficiency of the oxidative reactions and reforming.

Prior art processes generally use substantially higher severity of the reaction conditions compared to the present invention. The severity can be in the form of higher temperature, higher pressure or higher concentrations of solvents or oxidizing agents such as pure O₂ or other costly oxidizers. For example, concentration of solvents in reviewed prior art ranges from 0.12 to 10 times the weight of feedstock. The present invention can be used to lower overall process costs and allows the commercialization of chemicals from coal and similar carbonaceous feedstocks, which has not been achieved before. Furthermore, the extent of conversion or oxidation can be controlled in the processes of the present invention to yield different types and amounts of partial conversion or oxidation products. Further, the process conditions of the present invention can be adjusted to eliminate converted coal solids, other than inorganics concentrated in an ash stream, without significant loss to CO₂.

Further, the present invention is directed to a process for treatment of black liquor to produce significant amounts of small organic compounds of various types. The treatment comprises a step of treating black liquor with an oxidizing agent to generate one or more organic compounds comprising from about 2 to about 20 carbon atoms.

The term “black liquor” as used herein has its ordinary meaning in the pulp and paper industry. The term “black liquor” also refers to the liquor resulting from the cooking of pulpwood in an alkaline solution in a soda or sulfate, such as a Kraft, paper making process by removing lignin, hemicelluloses, tall oil, and other extractives from the wood to free the cellulose fibers.

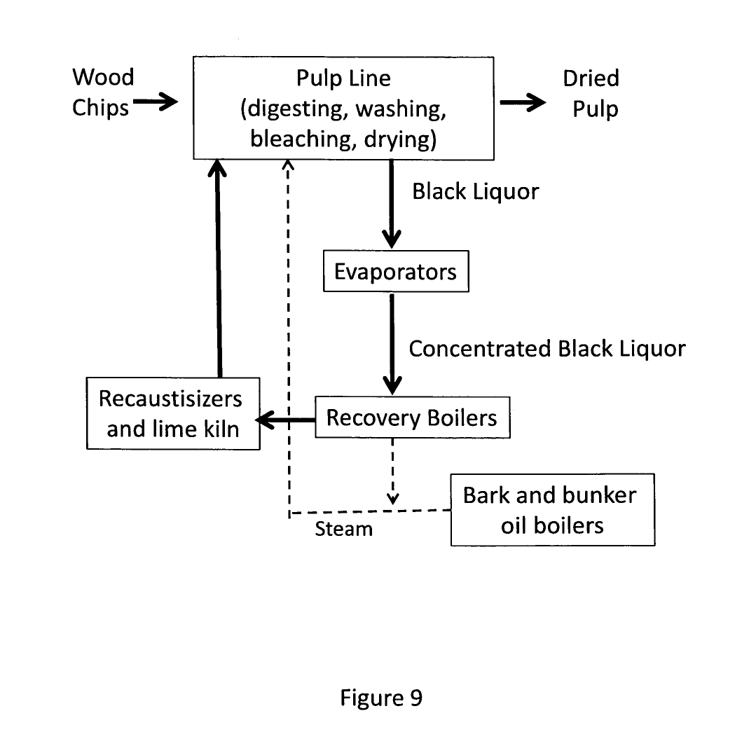

FIG. 9 presents a flow chart that shows a prior art process practiced by many pulp mills in producing black liquor, treating the black liquor and recovering energy from the black liquor. The present invention acts on the black liquor after it is recovered from the pulping process and prior to the conventional step of energy recovery by burning.

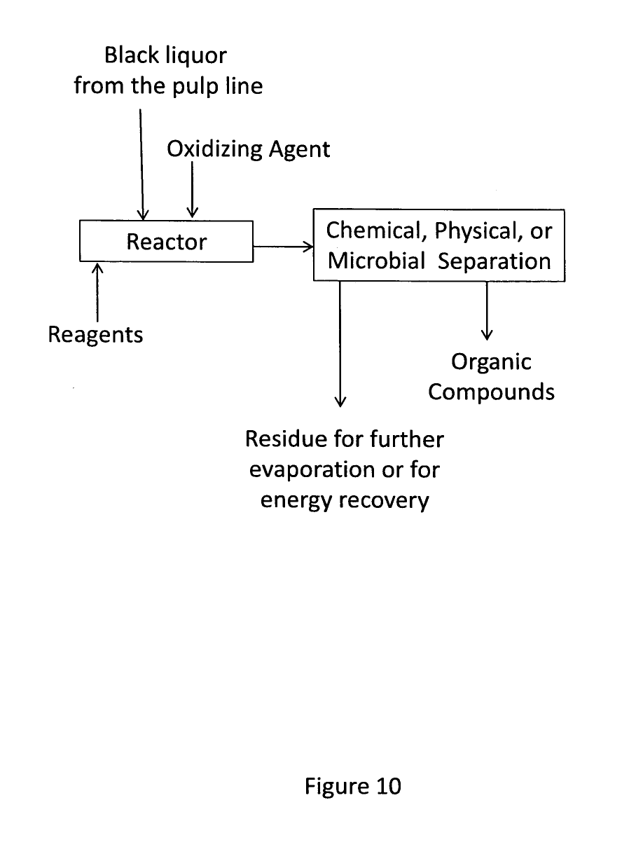

One embodiment of the treatment of black liquor by the process of the present invention is exemplified in FIG. 10. The black liquor and an oxidizing agent are fed into the reactor, along with optional additional reagents, and heated under pressure. The reaction within the reactor creates a reaction mixture, which can then be treated and/or separated by chemical, physical or microbial means, to yield organic compounds. These organic compounds include organic compounds comprising from about 2 to about 20 carbon atoms.

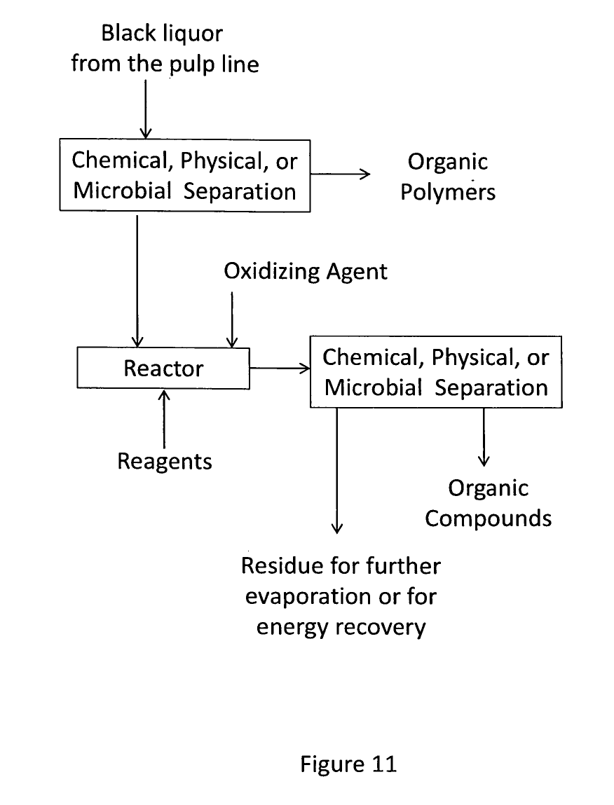

In alternative embodiments, the black liquor is separated, or fractionated, into various components prior to the treatment. One possible embodiment is exemplified in FIG. 11. The black liquor is separated by a chemical, a physical or a microbial process, selected organic polymers are recovered as an economically valuable commodity, and the balance of the black liquor is a black liquor component reactor feedstock. This black liquor component reactor feedstock is fed, along with an oxidizing agent and optional additional reagents, into the reactor, and is heated under pressure. The reaction within the reactor creates a reaction mixture, which can then be treated and/or separated by chemical, physical or microbial means, to yield organic compounds. These organic compounds include organic compounds comprising from about 2 to about 20 carbon atoms.

The advantage of the embodiment exemplified in FIG. 11 is that those organic polymers which are economically valuable may be sold to obtain a greater profit than the organic compounds that are generated by the reactor and subsequent chemical, physical, or microbial separation.

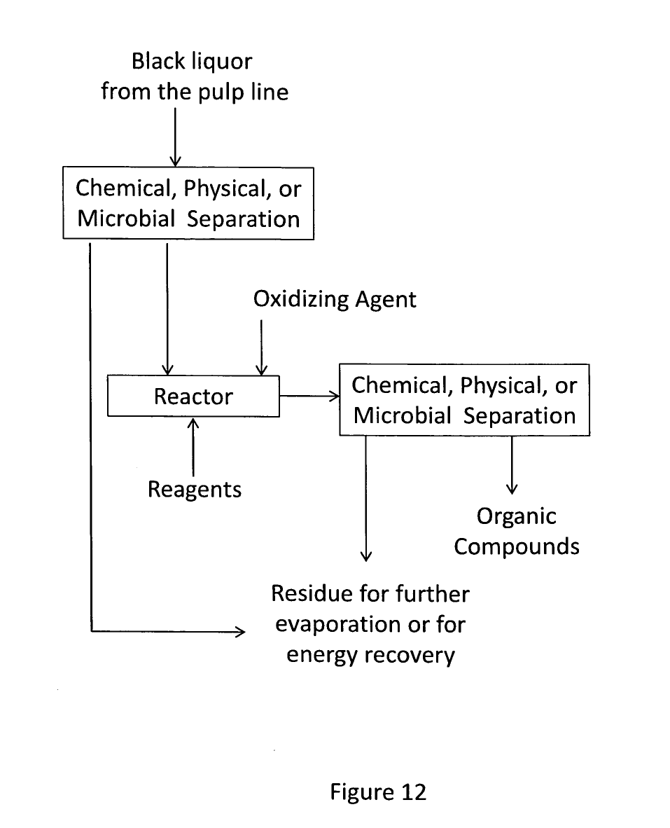

In another alternative embodiment the black liquor is separated, or fractionated, into various components prior to the treatment, as exemplified in FIG. 12. The black liquor from the pulp line (“raw black liquor”) is separated by a chemical, physical or microbial process, to obtain the black liquor component reactor feedstock, and a residue. The black liquor component reactor feedstock is fed, along with an oxidizing agent and optional additional reagents, into the reactor, and is heated under pressure. The reaction within the reactor creates a reaction mixture, which can then be treated and/or separated by chemical, physical or microbial means, to yield organic compounds. These organic compounds include organic compounds comprising from about 2 to about 20 carbon atoms. The residue from the separation of raw black liquor is further dewatered, and burned in a recovery boiler to produce energy.

The advantage of the embodiment exemplified in FIG. 12 is that the separation of the black liquor form the pulp line prior to feeding to the reactor is that the black liquor is cleaned up to get rid of components which decrease yield, efficiency, or profitability of the process to make organic compounds comprising from about 2 to about 20 carbon atoms. These undesirable components may then still be useful as a fuel.

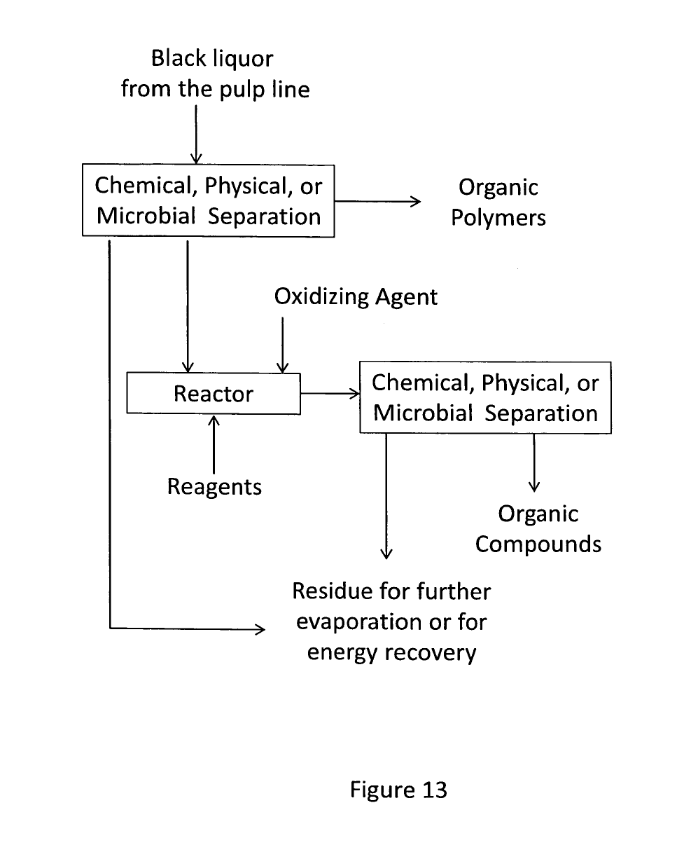

In still another alternative embodiment the black liquor is separated, or fractionated, into various components prior to the treatment, as exemplified in FIG. 13. The black liquor is separated by a chemical, physical or microbial process, to obtain the selected organic polymers, the black liquor component reactor feedstock, and the residue. The black liquor component reactor feedstock is fed, along with an oxidizing agent and optional additional reagents, into the reactor, and is heated under pressure. The reaction within the reactor creates a reaction mixture, which can then be treated and/or separated by chemical, physical or microbial means, to yield organic compounds. These organic compounds include organic compounds comprising from about 2 to about 20 carbon atoms. The residue from the separation of raw black liquor is further dewatered, and burned in a recovery boiler to produce energy.

The advantage of the embodiment exemplified in FIG. 13 is that the separation of the raw black liquor into three streams (i.e., the organic polymers, the black liquor component reactor feedstock, and the residue) is that by balancing the contents of the three streams may optimize the process to achieve highest return on investment.

The composition of the black liquor component reactor feedstock may be adjusted to obtain a better quality reactor feedstock. Such a better quality reactor feedstock may improve yields of a particularly commercially valuable organic compound; or it may result in composition that reacts faster, easier or more cheaply than the black liquor from the pulp line; or the chemical, physical or microbial separation may be made easier.

The black liquor component reactor feedstock comprises a mixture of water, and organic solids. The black liquor component reactor feedstock may optionally also comprise inorganic solids. The black liquor component reactor feedstock has different composition from the composition of the black liquor from the pulp line. Specifically, it is lower in whatever contents are separated from the black liquor, such as some organic polymers (in case of the embodiment exemplified by FIG. 11), residue for further evaporation and energy recovery (in case of the embodiment exemplified by FIG. 12), or both (in case of the embodiment exemplified by FIG. 13).

Such separation of components from the raw black liquor may decrease the concentration of some components, and thus increase the relative concentration of other components. For example, removal of soaps and/or tall oils, will increase the concentration of the lignin. Under one embodiment the concentration of lignin is increased from about 35 to 45 wt % with respect to the total organics to at least 55 wt %. Under another embodiment, the concentration is increased to at least 65 wt %. Under another embodiment, the concentration is increased to at least 75 wt %.

The term “lignin” means a phenylpropane polymer of amorphous structure including about 17 to about 30%, by weight, wood. Lignin can be associated with holocellulose that can make up the balance of a wooden material separated by conducting a chemical reaction at a high temperature. Generally, although not wanting to be bound by theory, it is believed that lignin serves as a plastic binder for holocellulose fibers.

The definition of the term “cellulose” includes a natural carbohydrate-high polymer, e.g., polysaccharide, including anhydroglucose units joined by an oxygen linkage to form long molecular chains that are essentially linear. The degree of polymerization can be about 1,000 units for wood pulp to about 3,500 units for cotton fiber with a molecular weight of about 160,000-about 560,000.

The term “hemicellulose” means cellulose having a degree of polymerization of 150 or less.

The term “holocellulose” means the water-insoluble carbohydrate fraction of wood.

The term “tall oil” refers to a mixture of rosin acids, fatty acids, and other materials obtained by an acid treatment of alkaline liquors from digesting or pulping of woods, such as pine. Moreover, the spent black liquor from the pulping process can be concentrated until the sodium salts, such as soaps, of the various acids can be separated and then skimmed off. These salts can be acidified by sulfuric acid to provide additional tall oil. The composition can vary widely, but can, for example, average about 35 to about 40%, by weight, rosin acids and about 50 to about 60%, by weight, of fatty acids.

The present invention provides a method of converting at least part of a black liquor feedstock to converted products and biodegradable substrates. The invention can simultaneously or serially oxidize, depolymerize, reform and/or solubilize low-valued high molecular weight materials in the black liquor feedstock to lower molecular weight hydrocarbons and oxygenated organic compounds, as well as other low molecular weight compounds.

The phrase “oxygenated organic compound” refers to an organic compound that comprises at least one oxygen atom. Examples of the oxygenated organic compounds include oxygenated hydrocarbons, and oxygenated compounds comprising additional heteroatoms.

The term “heteroatom” means any atom besides hydrogen or carbon. Examples of heteroatoms include oxygen, nitrogen, phosphorus, sulfur, fluorine, and chlorine.

Examples of oxygenated hydrocarbons include alcohols, aldehydes, carboxylic acids, salts of carboxylic acids, esters, ethers, anhydrides, and like. Oxygenated compounds may be monofunctional, difunctional, trifunctional, or polyfunctional. Included in the definition of oxygenated hydrocarbons are also compounds with more than one functional group, such as polyols, dicarboxylic acids, triacids, polyesters, polyethers, aldehydic acids, and like. Included in the definition of oxygenated hydrocarbons are also compounds in which there is more than one functional group wherein the functional groups are different.

Examples of carboxylic acids include compounds of the formula R—COOH, wherein R is an alkyl group. Particular examples include formic or mathanoic acid, acetic or ethanoic acid, propionic acid, butyric acid, butanoic acid, valeric acid, pentanoic acid, caproic acid, hexanoic acid, enanthic acid, heptanoic acid, caprylic acid, octanoic acid, pelargonic acid, nonanoic acid, capric acid, decanoic acid, undecylic acid, undecanoic acid, lauric acid, dodecanoic acid, tridecylic acid, tridecanoic acid, myristic acid, tetradecanoic acid, pentadecanoic acid, palmitic acid, hexadecanoic acid, margaric acid, heptadecanoic acid, stearic acid, octadecanoic acid, arachidic acid, and icosanoic acid.

Dicarboxylic acids of the present invention are organic compounds that contain two carboxylic acid groups. Such dicarboxylic acids may comprise additional heteroatoms, such as oxygen, nitrogen, or sulfur. Dicarboxylic acids may be aliphatic or aromatic. Aside from the two —COOH groups, dicarboxylic acids may be saturated or unsaturated. The dicarboxylic acids may be represented by the formula HOOC—R—COOH, wherein R is a difunctional organic group, such as alkylene, alkenylene, alkynylene, arylene, and any of the preceding modified by a one or more heteroatoms.

Examples of dicarboxylic acids include compounds such as alkylene dicarboxylic acids, having the general formula HOOC—(CH₂)ₙ—COOH wherein n is 0 to 12; mono-unsaturated forms thereof; di-unsaturated forms thereof; tri-unsaturated forms thereof; and polyunsaturated forms thereof.

Examples of dicarboxylic acids include oxalic or ethanedioic acid, malonic or propanedioic acid, succinic or butanedioic acid, glutaric or pentanedioic acid, adipic or hexanedioic acid, pimelic or heptanedioic acid, suberic or octanedioic acid, azelaic or nonanedioic acid, sebacic or decanedioic acid, undecanedioic acid, and dodecanedioic acid.

Examples of aromatic dicarboxylic acids include phthalic acid, benzene-1,2-dicarboxylic acid, o-phthalic acid, isophthalic acid, benzene-1,3-dicarboxylic acid, m-phthalic acid, terephthalic acid, benzene-1,4-dicarboxylic acid, and p-phthalic acid.

Examples of monounsaturated acids include maleic acid, (Z)-butenedioic acid, fumaric acid, (E)-butenedioic acid, glutaconic acid, pent-2-enedioic acid, traumatic acid, and dodec-2-enedioic acid.

Example of di-unsaturated acids includes three isomeric forms of muconic acid, and (2E,4E)-hexa-2,4-dienedioic acid.

An exemplary reaction of the present invention resulted in a reaction mixture that includes a variety of small organic molecules, including succinic acid (2.49%), malic acid (0.59%), fumaric acid (0.36%), glutaric acid (0.19%), propane 1,2,3-tricarboxylic acid (0.15%), and heptanoic acid (0.10%). See FIG. 3 for a GCMS spectrum of the acid fraction of this exemplary reaction of the present invention.

The identity and amounts of small organic compounds in the reaction product depends on the treatment parameters, such as the reaction conditions including the pressure, and reaction temperature, the type of oxidant used, and the weight ratios of the oxidant to the black liquor. In one embodiment of the present invention, the treatment of the black liquor yields primarily alcohols and ethers. In another embodiment of the present invention, involving further oxidation, the reaction product comprises greater relative amounts of aldehydes. By increasing the degree of oxidation further, the reaction product may comprise greater relative amounts of carboxylic acids and esters.

The alcohols, ethers, aldehydes, esters, and carboxylic acids may be monofunctional, or polyfunctional. For example, the treatment of the black liquor by the method of the present invention may result in mono-, di-, and tricarboxylic fatty acids.

In one embodiment, the black liquor may be heated in a reaction vessel in the presence of at least one oxidizing agent. The treating step may comprise raising the temperature of the mixture to a desired temperature by any suitable means and/or subjecting the mixture to a pressure at or above the steam saturation pressure. Multiple reactions may occur during the treatment step, including oxidation, depolymerization, reforming and solubilization. In a reforming process, the molecular structure of a hydrocarbon is rearranged. Without being bound by theory, it is believe that the treatment step of the present invention may oxidatively crack wood polymers to provide small organic compounds.

The oxidizing agent may be selected from air, oxygen enriched air, ozone, sulfuric acid, permanganates, carbon dioxide, nitrous oxide, nitric acid, chromates, perchlorates, persulfates, superoxides, chlorates, peroxides, hypochlorites, Fenton's reagent and nitrates in which the cations may comprise metal cations, hydrogen ions and/or ammonium ions.

Oxidizing agents may be ranked by their strength. See Holleman et al. “Inorganic Chemistry,” Academic Press, 2001, page 208. A skilled person will appreciate that, to prevent over-oxidation of the carbonaceous materials, the conditions in the treatment step may be adjusted according to the strength of the oxidizing agent used. For example, when a strong oxidizing agent is used, one or more of temperature, pressure, and duration of the treatment step may be reduced to prevent over-oxidation and/or ensure that the desired degree of conversion is not exceeded. On the other hand, when a weak oxidizing agent is used, one or more of temperature, pressure, and duration of the treatment step may be increased to ensure that the desired degree of oxidation and/or conversion is achieved. When the oxidizing agent is gaseous, the pressure in the reaction vessel for the treatment step is important for ensuring the desired degree of oxidation and/or conversion.

In some embodiments, oxygen is used as the oxidizing agent. In one embodiment, oxygen can be delivered to the reaction vessel as air. In some other embodiments, depending on the susceptibility of the carbonaceous feedstock to oxidation, oxygen-enriched air can be used. Suitable enrichment percentages can provide an oxygen concentration slightly above that of atmospheric air to a concentration equivalent to substantially pure oxygen.

The black liquor stream as generated by the pulping process is typically very caustic. Such a caustic environment is typically sufficient to allow oxidative cracking of wood polymers to generate one or more organic compounds comprising from about 2 to about 20 carbon atoms. However, in some cases, the black liquor stream may have a lower pH that does not readily allow for acceptable oxidative cracking of wood polymers to generate one or more organic compounds comprising from about 2 to about 20 carbon atoms. Under such circumstances, a mineral base may be added to the black liquor. Exemplary bases that may be used include Group I (alkali metal) and Group II (alkaline earth) oxides, hydroxides, carbonates, borates, and halogenates. In particular, sodium, potassium, calcium, and magnesium compounds are preferred. Examples of suitable bases include sodium hydroxide and potassium hydroxide.

Naturally occurring minerals may also be helpful in aiding oxidation. Examples of such minerals include, nahcolite, trona, thermonatrite, gaylussite, hydromagnesite, lansfordite, ikaite, hydrocalcite, dolomite, huntite, aragonite, natrite, magnesite, calcite, kalcinite, and gregoryite.

The mineral bases generally comprise no more than 15 wt % of the mixture provided to the treatment step, and preferably comprise below 10 wt % and most preferably at or below 6 wt % of the mixture provided to the treatment step. In some embodiments, the base comprises at least 1 wt % or at least 3 wt % or at least 5 wt % of the mixture fed to the treatment step.

In alternative embodiments, depending on the target small organic molecules sought, instead of using base, a mineral acid may be used to provide more acidic conditions for carrying out the reaction. Examples of suitable mineral acid include phosphoric acid, nitric acid, boric acid, hydrochloric acid, and sulfuric acid.

In some embodiments, at least one catalyst may optionally be added to the mixture. The catalyst may catalyze the oxidation reaction by, for example, causing or enhancing formation of peroxides and superoxides, which may enhance the rate of oxygen insertion into the carbonaceous material relative to oxidation of the black liquor in the absence of such catalysts.

The catalyst may be selected from water insoluble metals, transition metals, and precious metals. Examples of these metals include nickel, cobalt, platinum, palladium, rhenium, copper, vanadium and ruthenium. The catalyst may be unsupported or may be supported on an inert or active matrix material such as clay, alumina, silica, silica alumina, zeolites, activated carbon, diatomaceous earth, titania, zirconia, molybdena, ceramics, and the like. Such catalysts can enhance rates of oxygen insertion and reforming of high molecular weight carbonaceous compounds as well as being able to enhance the degree of relative oxidation. Examples of the catalysts include metal oxides, mixed metal oxides, hydroxides, and carbonates, of ceria, lanthanum, mixed rare earths, brucite, hydrotalcite, iron, clays, copper, tin, and vanadium.

The reaction vessel in which the treatment step is conducted is not limited to any particular reactor design, but may be any sealable reaction vessel that can tolerate the temperature and pressure required for the present invention. In some embodiments, the mixture is fed to a reactor, which has been pre-heated to the desired temperature. Then, air or oxygen enriched air is slowly added to the reactor until the desired pressure is reached. The temperature and pressure in the reactor may be monitored during the filling of air or oxygen enriched air, as well as during the treatment step itself.

The treatment of the black liquor according to the present invention occurs at a temperature sufficient to oxidize components of the black liquor to generate one or more organic compounds comprising from about 2 to about 20 carbon atoms. This temperature has been found to be up to about 300° C., or between about 150° C. and about 250° C. In another embodiment, the treatment of the black liquor occurs at a temperature between about 150° C. and about 220° C. In yet another embodiment, the treatment of the black liquor occurs at a temperature below about 150° C.

Treatment of the black liquor according to the present invention occurs at a pressure sufficient to oxidize components of the black liquor to generate one or more organic compounds comprising from about 2 to about 20 carbon atoms. This pressure has been found to be below about 1230 psig or about 322 psig. In another embodiment, this pressure has been found to be below about 54 psig. In certain embodiments, this pressure ranges from atmospheric pressure to about 1230 psig, or about 322 psig or about 54 psig.

The duration of the treatment step may be determined, for example, by the oxidative stress induced in the mixture and the desired product. As a general rule, a higher oxidative stress requires a shorter duration treatment step. In addition, if the desired products are generated by more complete oxidation of the carbonaceous materials, e.g., via a series of sequential reaction steps, a longer duration treatment step may be required.

Reaction times can vary from a few seconds to several hours, depending on the degree of conversion and/or oxidation required, the reduction in molecular weight desired, the reactivity of the feedstock, the type and/or amount of oxidizing agent employed, whether a catalyst is employed, process economics, the amount of carbon dioxide, carbon monoxide, and hydrogen generated, and other constraints. Exemplary reaction times range from about 0.5 to about 4 hours, or about 1 to about 3 hours, or about 2 hours.

In some embodiments, the reaction conditions including temperature, pressure and reaction time may also depend on the molecular and elemental characteristics of the particular black liquor feedstock. Different species of wood may result in differing compositions of the black liquor. The characteristics of the black liquor used in the pulping process which may need to be taken into consideration are the degree of aromaticity, the hydrogen to carbon ratio, the oxygen to carbon ratio, the nitrogen to carbon ratio, the sulfur to carbon ratio, and the mineral or ash content, as well as other factors.

The small organic compounds generated by the treatment step may be separated and isolated from reaction mixture. Applicable chemical and physical separation technologies that may be used include any of those known to one skilled in the art, including fractional distillation, liquid/liquid extraction, adsorption, ion exchange, membrane filtering, and hybrid systems. In one embodiment of the present invention, the separation may be achieved in a similar fashion that is used to separate tall oils (saponification and salting out).

An alternative to recovering the reaction products via physical or chemical separation after the completion of the treatment step, involves subjecting the reaction products to microbial digestion. The reaction products may be introduced to a microbial digester, where the reaction products may undergo a bioconversion process. During the bioconversion process, some, or all, of the reaction products may digested by one or more microorganisms present in the microbial digester. In one embodiment, the bioconversion process may produce biogases such as methane, hydrogen, carbon monoxide, and other gases and mixtures thereof, which may be used as fuel or can be converted to electricity.

The conditions in the microbial digester may be optimized to achieve a high degree of biodegradation of the reaction products, including controlling one or both of the degree and rate of bioconversion. The reaction products obtained from the treatment step may affect one or both of the degree and rate of bioconversion in bioconversion process. Thus, in one aspect of the invention, the conditions of the treatment step are selected on the basis of producing reaction products that may include larger quantities of biodegradable materials and/or may exhibit an enhanced rate of biodegradation or an enhanced tendency to biodegrade when subjected to a subsequent bioconversion step.

Upon separation of selected organic compounds from the resulting reaction products from the treated black liquor, a residue is obtained. The residue may then be handled as is routinely done by pulp mills today, such as burning it in the boiler for energy recovery.

### EXAMPLES

The following examples are illustrative, but not limiting, of the methods of the present disclosure. Other suitable modifications and adaptations of the variety of conditions and parameters normally encountered in the field, and which are obvious to those skilled in the art, are within the scope of the disclosure.

### Example 1

Coal or other carbonaceous feedstock was wet milled to provide an aqueous slurry with a median particle size of about 20 μm. The slurry was then fed to a continuous stirred-tank reactor (CSTR), operated in a batch or continuous mode. An alkali base such as NaOH was added to the aqueous slurry. O₂ was introduced to the CSTR via pressurization of the headspace with compressed air or O₂-enriched air in batch mode, or via a continuous flow of air for continuous mode. Solids content, alkali base concentration, temperature, pressure, and stirring rate were adjusted to achieve various degrees of oxidative depolymerization of the carbonaceous feedstock.

### Example 2

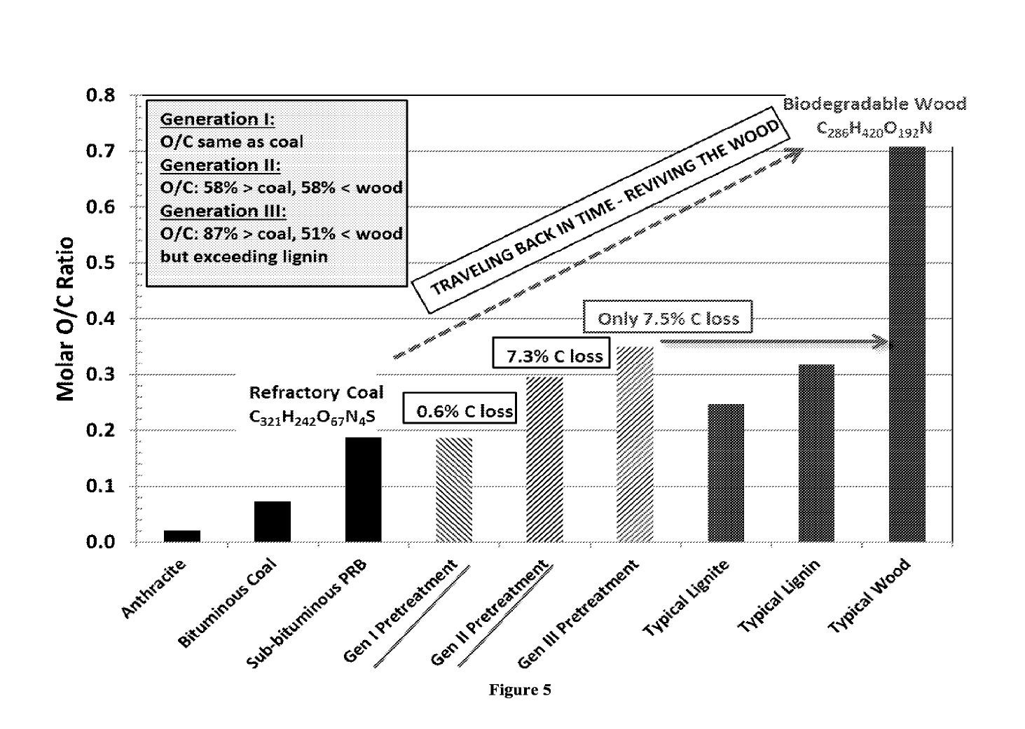

In this example, coal was treated using three different methods: Generations I, II and III. The methods of the present invention were able to increase the oxygen/carbon (O/C) ratio of the coal due to oxidation of the carbonaceous materials in the coal. The degrees of oxygenation varied after different generations of pretreatments; relative to other common carbonaceous materials (FIG. 5). For this example, Generation I, H or III pretreatments were the same as Example 1, except for the conditions noted here. Generations I and II had an operating temperature of 230° C. while Generation III was heated to 155° C. The mixture used in all of the three embodiments had a coal content of 20% by weight in the reactor, and an amount of NaOH to provide 6% by weight, based on the weight of the coal. The pressure in the headspace of the reactor was atmospheric, 400 psig, or 800 psig for Generations I, II and III, respectively. The hold time was 0.5 hour for oxidation of the carbonaceous materials.

The degrees of oxygenation, represented by molar O/C ratios, were calculated from headspace gas analysis before and after the experiment, resulting in retention of O₂ in the coal. O₂ retention was also verified by ultimate analysis (C,H,O) of the treated slurry, in comparison with the coal before the treatment. The carbon losses shown on the graph were calculated in the same fashion. Molecular formulae of coal and wood, as well as, O/C ratios for various feedstocks were obtained from reported literature.

The O/C ratios of the treated coal and other carbonaceous feedstocks are represented in FIG. 5. Generation I treatment did not change the O/C ratio for the coal significantly, with only 0.6% carbon loss due to the treatment. Generation II treatment increased the O/C ratio of the coal by 58%, with a carbon loss of 7.3%. The final O/C ratio of the coal after the Generation II treatment is still 58% lower than a typical wood. Generation III treatment increased the O/C ratio of the coal by 87%, with a carbon loss of 7.5%. The final O/C ratio of the coal after the Generation III treatment is about 51% lower than a typical wood.

It is expected that higher extents of oxygenation may be achieved by increasing the pressure in the headspace or lengthening the contact time in order to provide a higher O/C ratio for the treated coal. This will bring the O/C ratio of the treated coal towards the O/C ratio of biodegradable wood. From this example, it appears that the method of the present invention is able to oxygenate coal to make it more biodegradable.

### Example 3

In another example, the correlation between oxygen retention and starting oxygen content in the headspace of the reactor, with or without catalyst, was studied. The procedure was similar to Example 1, with a reaction temperature of 145° C. and solids content of 10% in the reactor. Headspace pressure was varied from 100 to 1300 psig to achieve different starting O₂/coal ratios (starting oxygen). O₂ retained was again calculated from headspace analysis by a gas chromatograph (GC), and verified by ultimate analysis (C,H,O) of the treated slurry.

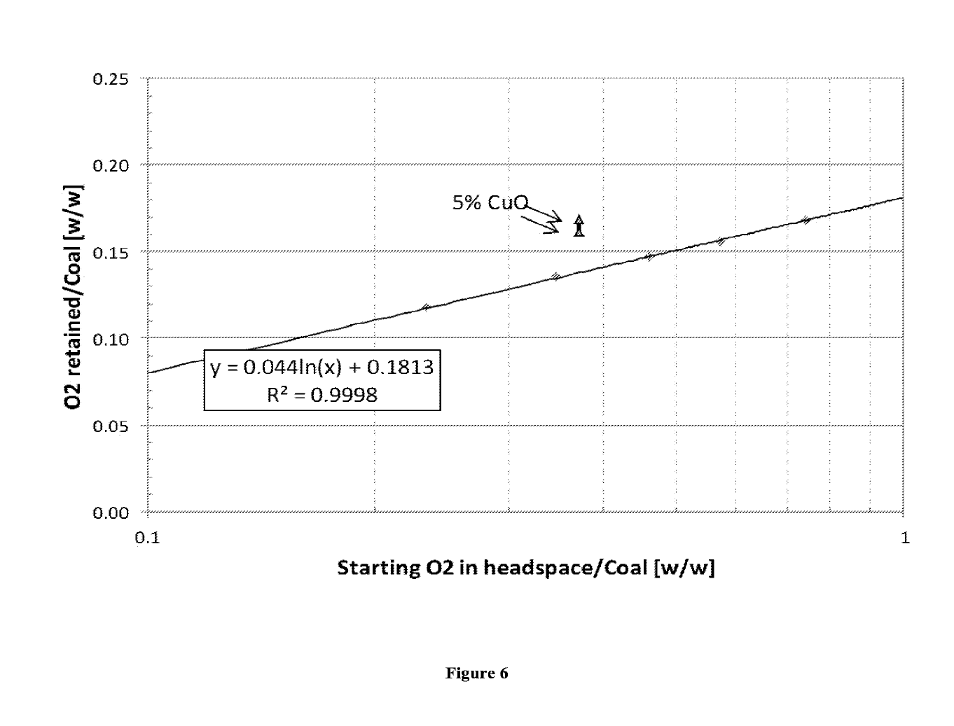

The efficiency of oxygen retention in coal was dependent on the amount of oxygen available for oxidation in the headspace (FIG. 6). When a metal oxide catalyst such as CuO was added to the reaction mixture, the retention efficiency was significantly increased. Here 5% CuO (wt/wt coal) was used, leading to a higher O₂ retention efficiency, thereby improving the effectiveness of the oxidation of coal.

### Example 4

In this example, the carbonaceous feedstock was subjected to two passes through the CSTR, in order to provide a more complete conversion of the coal to soluble carbon. The first pass was the same as in Example 3. For the 2^{nd} pass, the residual solids from the 1^{st} pass were subjected to the same conditions but half the amount of NaOH was used. Carbon conversions were calculated by measuring the concentration of dissolved organic carbon (DOC) in the treated slurry and CO₂ in the headspace (inorganic carbon or IC). Cake solids represent residual solids after the experiment and were measured by centrifuge followed by room temperature drying.

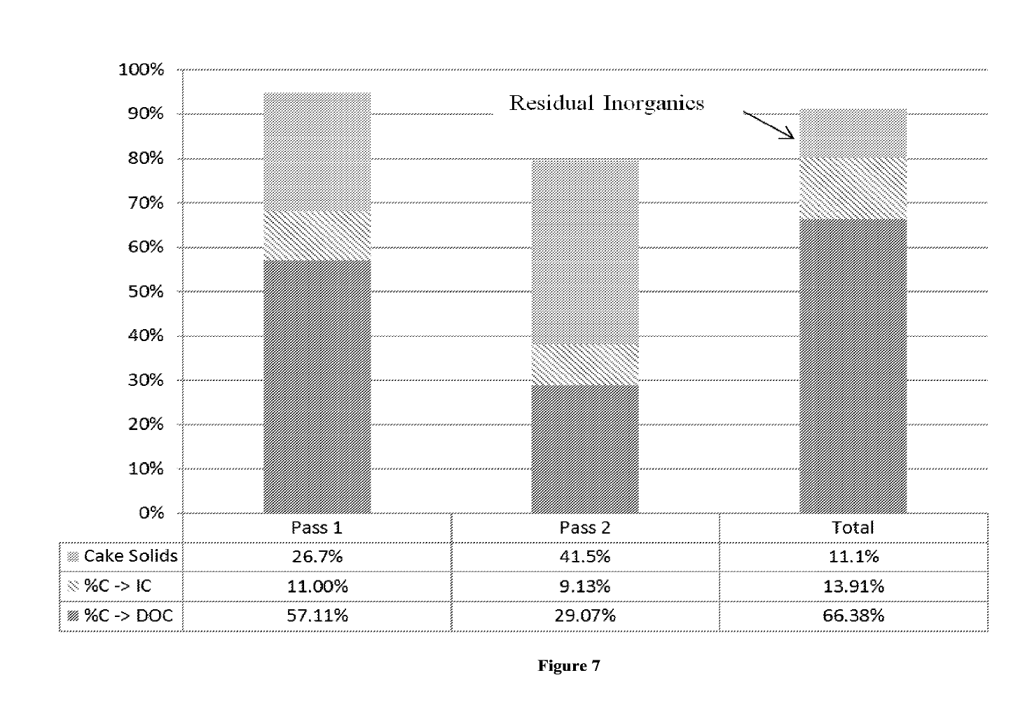

The carbon conversions after each of the first pass and second pass are presented in FIG. 7. The residual solids after the two passes were about 11.1% and very close to the ash content for this coal. About 66.4% of coal carbon was converted to DOC while only 13.9% was lost as CO₂. The 11.1% of coal solids that remained was comprised of mostly inorganics, and the ash content of this coal was about 9%. The example shows that essentially all organic carbon in this coal has been solubilized by two passes through the CSTR.

### Example 5

In this example, the reaction product from the CSTR was introduced to a microbial digester and the bioavailability of the carbonaceous materials was evaluated. The coal was treated using the procedure as described in Example 1 except that one treatment used 600 psig air in the headspace and a temperature of 120° C. (MM042512-R4) while the other treatment was carried out at 232° C. and using only atmospheric air in the headspace (MM051812-R4).

The treated coal was put into a microbial digester. A microbial culture was also added to the digester. The microbial culture was obtained from a wastewater processing facility. The growth of the microbial culture in the microbial digester represents the bioavailability of coal after the CSTR treatment. The microbial growth in the digester was measured at 0, 3 and 7-day time intervals. Cell growth was measured using the MPN technique at inoculation. The experiments were done in duplicate.

These experiments demonstrate that the treatment MM051812-R4 did not convert a significant proportion of the coal to biodigestible compounds, as there was insignificant microbial cell growth after 3 or 7 days, as compared with the starting point at 0 days. On the other hand, the treatment MM042512-R4 did convert a significant proportion of the coal to biodigestible compounds as evidenced by the growth of the microbial culture over the 7 day period, in comparison with the starting point at 0 days. The oxidative treatment (MM042512-R4), although conducted at lower temperature, provided products that resulted in a remarkably higher cell growth indicative of the higher bioavailability of the reaction products for microbial fermentation processes.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

### Example 6

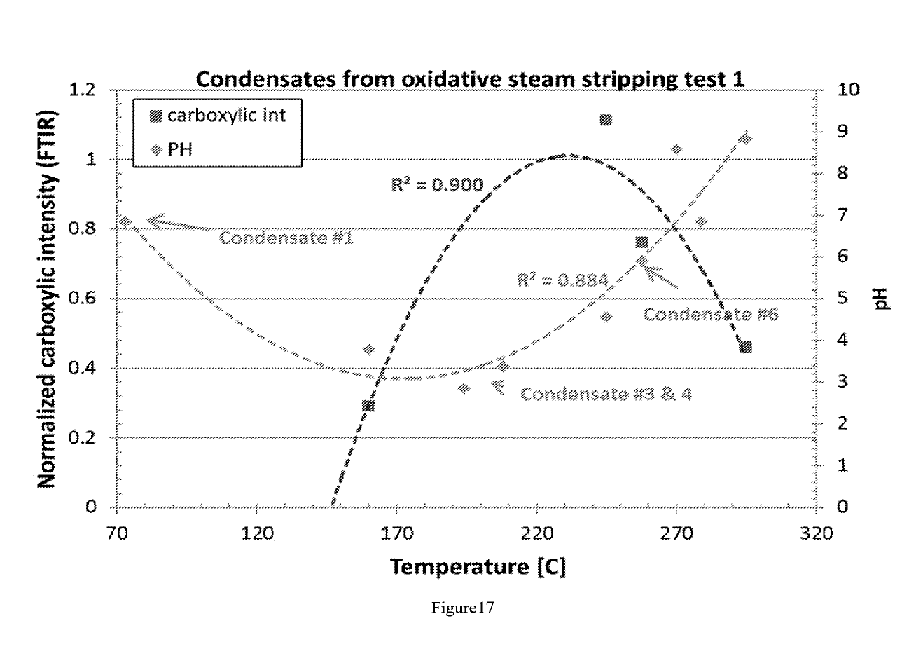

745 g of coal was placed in the fixed bed (column with 3″ diameter) and 100 g of water in the steam generator. Steam was generated at 230 degrees C. and air was provided at 300 psi and a flow rate of 13 L/min to generate a steam air mixture over the column of coal. This test continued for two hours during which the temperature of the fixed bed (at the wall) and gas composition leaving the bed were monitored. In addition, vapor products from the bed were condensed at 5 degrees C. and were analyzed by HPLC and GC-MS. Formation of carboxylic acids was followed by pH and FTIR, which indicated a maximum between 200-220 degrees C. based on the minimum pH and maximum intensity of the carboxylic peak in FTIR (see FIG. 17).

To measure the concentrations of volatile fatty acids (VFAs) produced, condensates 3 and 4 were analyzed by HPLC as shown in Table 1.

TABLE 1
VFA concentrations in mM

| | Formic | Acetic | Propionic | Butyric |
|---|---|---|---|---|
| Sample | Acid | Acid | Acid | Acid |
| Condensate 3 | 18.1 | 53.7 | 2.3 | 1.7 |
| Condensate 4 | 9.2 | 60.6 | 2.8 | 2.2 |
| Aqueous phase process | 27.4 | 19.4 | 0.0 | 0.0 |

The data in Table 1 show that the current process can shift the distribution to a significantly higher concentration of acetic acid which can then be separated from the mixture for marketing. Furthermore, the total concentration of these VFAs is about twice as much compared to the previous aqueous phase process. Off gases from this experiment contained CO2, N2 and O2.

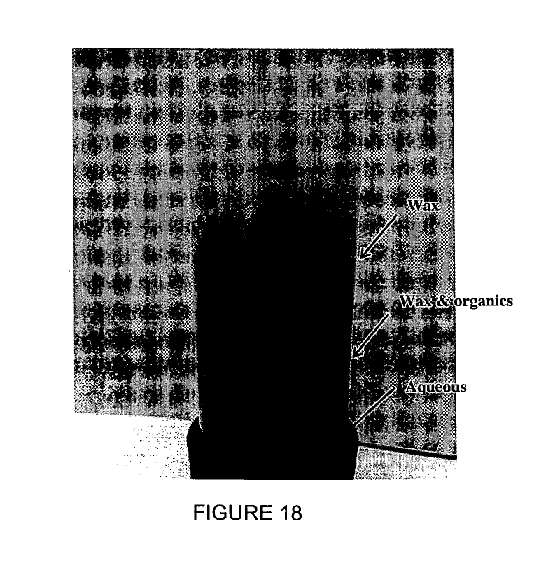

Beyond 220 degrees C. yield of carboxylic acids dropped and a hydrocarbon waxy phase started to appear in addition the aqueous phase and an organic phase. An image of this 3-phase product mixture is shown in FIG. 18 for condensate #6.

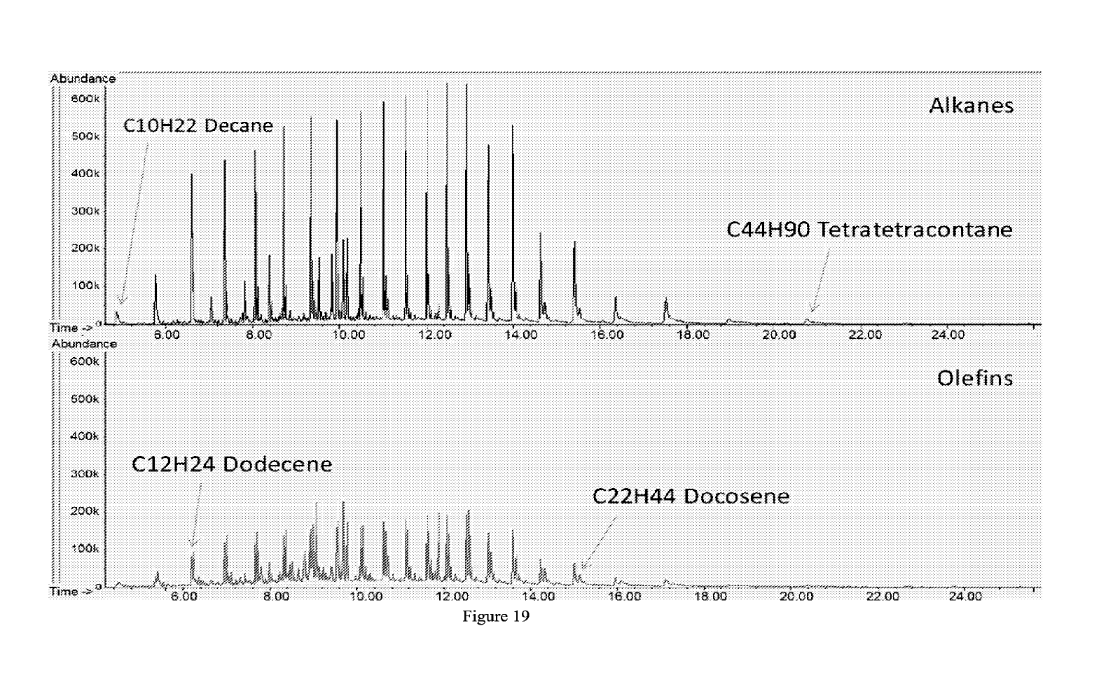

The waxy phase was extracted by hexane and was analyzed by GC-MS and resulted in the chromatogram in FIG. 19.

At higher temperatures it is believed that gasification is taking place as evidenced by the presence of small concentrations of CO and H2 in off gases, in addition to CO2, N2 and O2. However, at the same time, it appears that at least two other reactions namely water gas shift (WGS) and Fischer Tropsch (FT) are also taking place and possibly catalyzed by the presence of inorganic oxides of Co and Fe in the lignite.

### Example 7

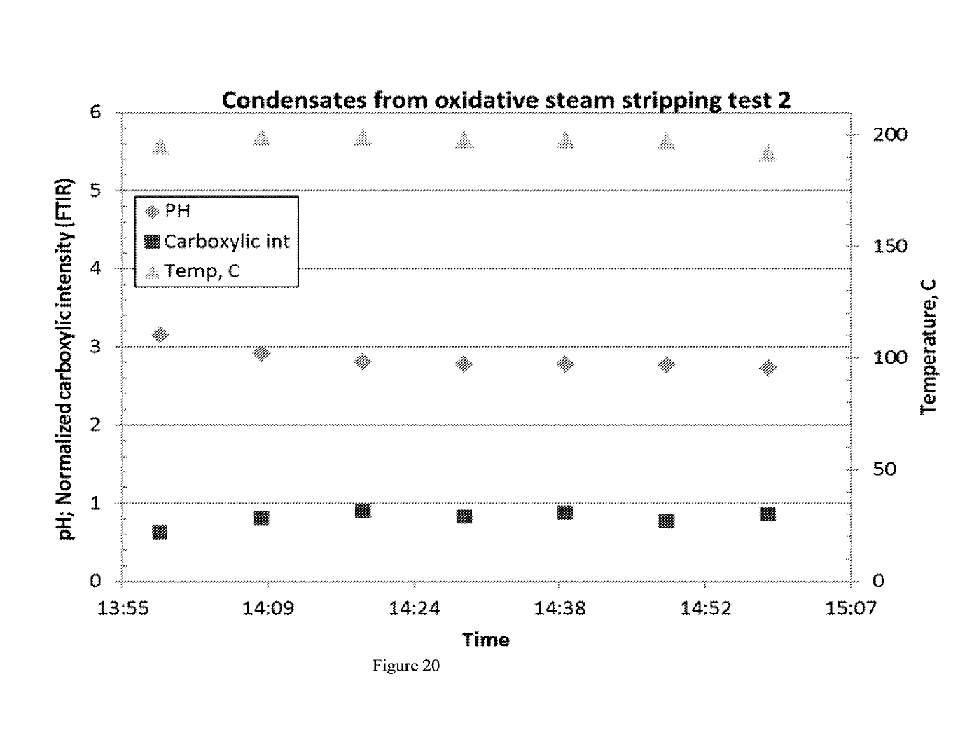

This test was performed at a relatively constant temperature of 200 degrees C. to stay in the partial oxidation regime where carboxylic acids are produced. The steady state time of this test was about 75 min during which vapor products from the bed were condensed at 5 degrees C. and were analyzed by HPLC and GC-MS. Formation of carboxylic acids was followed by pH and FTIR as shown in FIG. 20.

The concentrations of volatile fatty acids (VFAs) from a typical condensate from this experiment as analyzed by HPLC are shown in Table 2.

TABLE 2
VFA concentrations in mM

| | Formic | Acetic | Propionic | Butyric |
|---|---|---|---|---|
| Sample | Acid | Acid | Acid | Acid |
| Condensate | 20.5 | 8.2 | 1.2 | 0.0 |

### Example 8

This test was carried out in a different reactor configuration namely a continuous fluidized bed (4″ diameter) using low rank coal crushed and sieved to −50 mesh size. It was fed at the rate of 7.5 g/min. Bed temperature was 255 C and had a pressure of 2″ of water. Air was fed at 27.4 L/min without any steam. This flow rate satisfied the requirements of fluidization velocity as well as O2/coal needed for oxidative depolymerization. Steam was however generated in the bed from the inherent moisture in coal (about 40% moisture content of this coal). A cyclone and a filter downstream from the reactor captured any suspended fine coal particles and the resulting condensate was free of solids. Vapor products were condensed at 5 C and were analyzed by HPLC and GC. The condensate product had the following concentration of volatile fatty acids (VFA), in comparison with an aqueous process (Table 3).

TABLE 3
VFA concentrations in mM

| | Formic | Acetic | Propionic | Butyric |
|---|---|---|---|---|
| Sample | Acid | Acid | Acid | Acid |
| Condensate | 0 | 162.7 | 5.1 | 1.8 |
| Aqueous phase process | 27.4 | 19.4 | 0.0 | 0.0 |

It can be seen that a drastically higher concentration and selectivity towards acetic acid is achieved which makes this a valuable product mixture with low cost of separation.

### Example 9

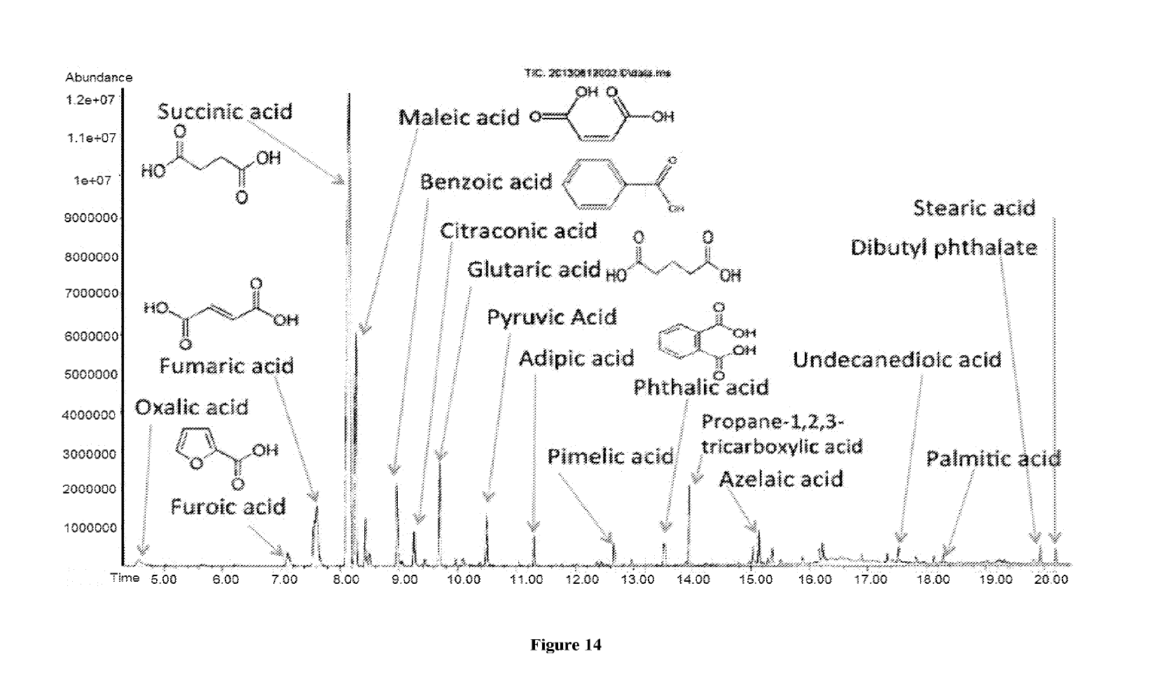

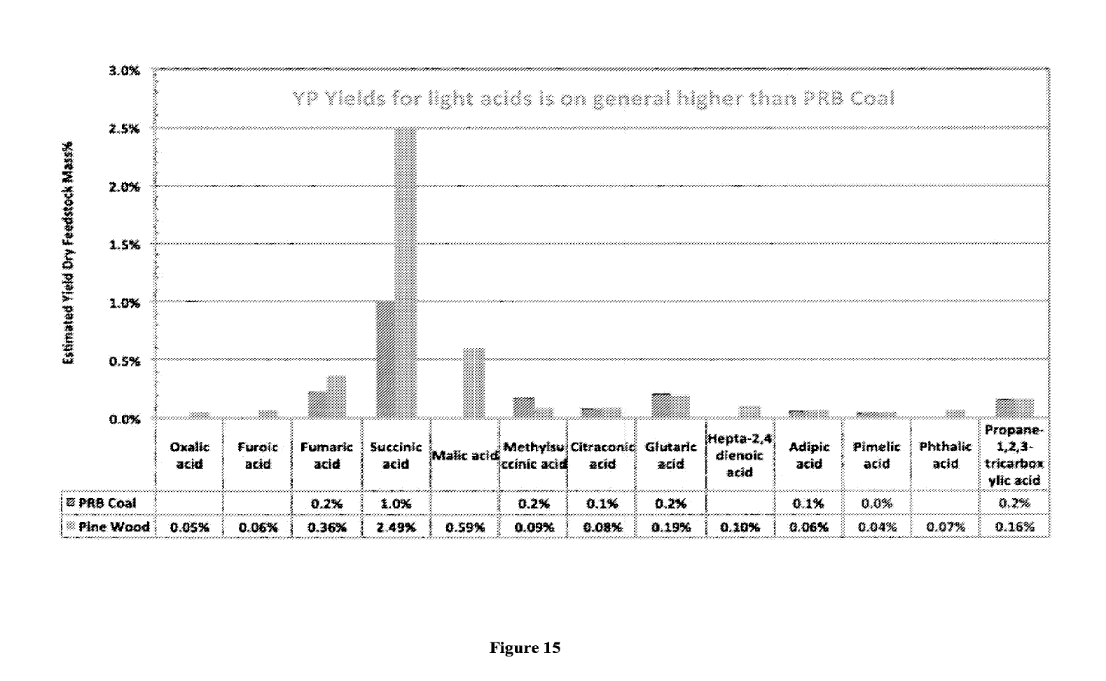

Shavings of pine wood were treated as described in the description above, to produce a reaction product. A gas chromatogram of the reaction product is shown in FIG. 14. A comparison of the product distribution of the reaction product obtained by the treatment step to a reaction product obtained from PRB coal is shown in FIG. 15.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

1. A method for treating a carbonaceous feedstock, comprising the step of heating a mixture of a carbonaceous feedstock with water in the presence of at least one oxidizing agent to a temperature below 300° C. and a pressure below 1230 psig.

2. The method of claim 1, wherein the mixture comprises at least one solubilizing agent selected from the group consisting of mineral acids or mineral bases.

3. The method of claim 1, wherein the heating step is configured as multiple heating steps and each heating step has at least one different condition selected from the group consisting of temperature, pressure, and duration.

4-6. (canceled)

7. The method of claim 2, wherein the mixture comprises at least one catalyst.

8. The method of claim 7, wherein the at least one catalyst is selected from the group consisting of non-soluble metals, transition metals and precious metals.

9. The method of claim 8, wherein the at least one catalyst is supported on a matrix material selected from the group consisting of clay, alumina, silica, silica alumina, zeolites, activated carbon, diatomaceous earth, titania, zirconia, molybdena, and ceramics.

10-13. (canceled)

14. The method of claim 1, wherein the at least one oxidizing agent is selected from the group consisting of air, oxygen enriched air, oxygen, ozone, perchlorates, carbon dioxide, nitrous oxide, oxides, superoxides, permanganates, chlorates, peroxides, hypochlorites, or nitrates.

15. The method of claim 1, wherein the at least one oxidizing agent comprises a cation selected from metal, hydrogen and ammonium ions.

16-22. (canceled)

23. The method of claim 1, further comprising a preprocessing step selected from grinding, milling, sieving or crushing the carbonaceous feedstock.

24. The method of claim 1, further comprising the steps of: separating at least one component from a product of the heating step by chemical and/or physical separation; and microbial digestion of the product of the heating step or the at least one separated component from the separating step.

25-27. (canceled)

28. The method of claim 24, wherein the microbial digestion step employs a microorganism or a microorganism consortium to digest carbonaceous materials in the product of the heating step.

29. (canceled)

30. The method of claim 24, wherein the microbial digestion step comprises a process selected from an aerobic process, an anaerobic process and combination of aerobic and anaerobic processes.

31. (canceled)

32. The method of claim 1, wherein the carbonaceous feedstock is selected from the group consisting of coal, lignite, tar sands, tars, crude oils, peat, pitch, resins, lignin, latex rubber, waxes, agricultural wastes, bark, wood, and algae cake.

33-35. (canceled)

36. A method for treating a black liquor or a component of black liquor, comprising a step of treating the black liquor or the component of black liquor with an oxidizing agent at a temperature of up to about 250° C. and a pressure of up to about 1230 psig to generate one or more organic compounds comprising from about 2 to about 20 carbon atoms.

37. (canceled)

38. The method of claim 36, wherein the one or more organic compounds comprise an oxygenated organic compound selected from the group consisting of an organic acid, an alcohol, an ester, an aldehyde, and an ether.

39-42. (canceled)

43. The method of claim 36, wherein a solubilizing agent selected from the group consisting of a mineral acids and a mineral base is present during the treating step.

44. The method of claim 36, wherein a catalyst is present during the treating step and the catalyst is selected from the group consisting of a non-soluble metal, a transition metal and a precious metal.

45. (canceled)

46. The method of claim 44, wherein the catalyst is supported on a matrix material selected from the group consisting of clay, alumina, silica, silica alumina, zeolite, activated carbon, diatomaceous earth, titania, zirconia, molybdena, and ceramics.

47-49. (canceled)

50. The method of claim 36, wherein the at least one oxidizing agent is selected from the group consisting of air, oxygen enriched air, oxygen, ozone, a perchlorate, carbon dioxide, an oxide, a superoxide, a permanganate, a chlorate, a peroxide, a hypochlorite, and a nitrate.

51-55. (canceled)

56. The method of claim 36, further comprising a step selected from the group consisting of a chemical separation, physical separation, and a microbial digestion carried out subsequent to the treating step.

57-62. (canceled)
